(19) United States
(12) Patent Application Publication
(10) Pub. No.: US 2026/0027258 A1
(43) Pub. Date: Jan. 29, 2026

(54) Antimicrobial Compositions

(71) Applicant: Matoke Holdings Limited, Abingdon, Oxfordshire (GB)

(72) Inventor: Thomas Jon HALL, Abingdon, Oxfordshire (GB)

(73) Assignee: Matoke Holdings Limited, Abingdon, Oxfordshire (GB)

(21) Appl. No.: 18/998,581

(22) PCT Filed: Jul. 25, 2023
(86) PCT No.: PCT/GB2023/051957

§ 371 Date: Jan. 27, 2025

(30) Foreign Application Priority Data
Jul. 25, 2022 (GB) 2210839.3

(51) Int. Cl. A61L 26/00; C12N 9/04

(52) U.S. Cl. CPC A61L 26/0047; A61L 26/0019; A61L 26/0066; C12N 9/0006; C12Y 101/03004

(57) ABSTRACT

There is provided a liquid or gel composition for generating hydrogen peroxide. The composition comprises enzyme that is able to convert a substrate to release hydrogen peroxide; substrate for the enzyme; and polymer. The composition does not comprise sufficient free water to allow the enzyme to convert the substrate, has a water activity (aw) of 0.7 or less, and/or comprises 5% or less, by weight of water. The composition may have droplets of supersaturated aqueous solution comprising the substrate which are suspended in the polymer. There may be greater than 70%, by weight, of the polymer in the composition. The composition may comprise less than 2%, by weight, non-aqueous solvent, and i) greater than 50%, by weight, of the polymer, and/or ii) less than 20%, by weight, of sugar.

This invention relates to compositions for generating hydrogen peroxide, and their use in treating infections and wounds.

Honey has been used for treatment of microbial infections since ancient times. In recent years there has been a resurgence of interest in the therapeutic efficacy of honey, particularly in the area of wound healing. Clinical trials have shown that honey is an effective broad-spectrum antimicrobial agent which is effective against common wound-infecting organisms, such as Pseudomonas aeruginosa, Staphylococcus aureus, Candida albicans and Escherichia coli, and is effective against antibiotic-resistant strains of bacteria. As a natural product, honey also offers an attractive alternative to drug-based treatments.

Many different types of honey have antimicrobial activity. This activity is attributed largely to osmolarity, pH, hydrogen peroxide production and the presence of phytochemical components.

The applicant has appreciated that the antimicrobial effects of honey can be greatly enhanced and controlled by adding glucose oxidase to honey, and that compositions comprising honey and added glucose oxidase are applicable in the treatment of a number of infections, and notably in the treatment of infections caused by biofilms (see WO 2015/166197, WO 2016/083798 and WO 2016/124926).

However, because honey is a natural product, its composition can vary greatly depending on its source. For example, the difference in antimicrobial potency among honeys can be more than one hundred-fold, depending on the geographical, seasonal and botanical source of the honey, as well as the harvesting, processing and storage conditions. Consequently, the antimicrobial efficacy may also vary depending on the type of honey used. Furthermore, honey may also contain other components, such as allergens e.g. trace amounts of pollen, which may cause adverse reactions when applied to certain subjects and make it unsuitable for certain pharmaceutical applications. There is also considerable variability in physical characteristics such as viscosity and colour.

Honey is sticky and can be difficult to apply to, and remove from, a patient. Honey may also require processing such that it is in a suitable form for application to patients, which can add cost and complexity to the production process. Such processing may include creaming or pasteurisation.

Consequently, there is a desire to provide improved compositions which provide enhanced antimicrobial efficacy compared to honey, and which also overcome some of honey's disadvantages. There is also a desire to provide compositions with improved stability and which have the ability to provide antimicrobial activity over an extended period of time.

In a broad sense, the invention concerns hydrogen peroxide-generating compositions, particularly compositions comprising enzyme that is able to convert a substrate to release hydrogen peroxide, and substrate for the enzyme. Such compositions preferably comprise a thickening agent, such as polymer.

The invention also concerns methods of making hydrogen peroxide-generating compositions, in particular combining enzyme that is able to convert a substrate to release hydrogen peroxide and substrate for the enzyme. Such methods preferably include addition of a thickening agent, such as polymer.

Synthetic hydrogen peroxide-generating compositions are described in WO2018/065608A1; WO2020/193993A2 and WO2021/186165A1, and have included high substrate and sugar concentrations, or blends of polymer and non-aqueous solvent, and have included antioxidant. However, the applicant has surprisingly found that compositions with improved stability can be achieved without significant amounts of water, non-aqueous solvent and antioxidant, whilst requiring only low amounts of substrate. Such compositions maintain the ability to readily generate hydrogen peroxide on dilution, and have desirable texture and rheological properties to allow effective topical application.

According to the invention, there is provided a composition (preferably a liquid or gel composition) comprising: enzyme that is able to convert a substrate to release hydrogen peroxide; substrate for the enzyme; and polymer, wherein the composition: a) does not comprise sufficient free water to allow the enzyme to convert the substrate; and/or b) has a water activity (a_{w}) of 0.7 or less; and/or c) comprises 5% or less, by weight, of water, and wherein there is greater than 70% by weight of the polymer in the composition, and preferably 97% or less, by weight, of the polymer in the composition.

According to the invention, there is provided a method of preparing a composition (preferably a liquid or gel composition) comprising mixing: enzyme that is able to convert a substrate to release hydrogen peroxide; substrate for the enzyme; and polymer, wherein the composition is formulated such that: a) there is not sufficient free water to allow the enzyme to convert the substrate; and/or b) to have a water activity of 0.7 or less; and/or c) to comprise 5% or less, by weight, of water, and wherein the method comprises adding the polymer such that it is greater than 70%, by weight, of the composition, and preferably 97% or less, by weight, of the composition.

Preferably, the polymer is present in an amount of at least at least 80% by weight, even more preferably at least 90% by weight.

The polymer may comprise a plurality of polymers, or it may comprise only one polymer. So, if the polymer comprises 90% by weight polymer, this could include 45%, by weight, of a first polymer and 45% by weight of a second, different polymer.

The polymer may act as a thickening or gelling agent. The polymer may be, or may comprise, any medically acceptable polymer, such as any Food and Drug Administration-approved (FDA-approved) polymer.

The polymer may be, or may comprise, a synthetic polymer. In some embodiments, the polymer may be or may comprise a natural polymer, or biopolymer, such as a polysaccharide or a polypeptide. The polymer may be a hydrocolloid.

Preferably, the polymer is, or comprises, a water-soluble synthetic polymer. In preferred embodiments, the polymer is or comprises a polyether, such as polyethylene oxide (or polyethylene glycol (PEG)). Other suitable polymers may include polyvinyl alcohol, polyvinylpyrrolidone, poly(lactic-co-glycolic acid), polyglycolic acid, polylactic acid, polycaprolactone, polymeric surfactants, polyacrylic acid, polyacrylamide, N-(2-hydroxypropyl) methacrylamide (HPMA), polyoxazolines, polyphosphates or polyphosphazenes. Another suitable polymer may be phosphino-carboxylic acid (PCA).

The polymer may be, or may include, a polypeptide or a polysaccharide such as cellulose (which includes derivatives such as hydroxypropyl methyl cellulose and hydroxypropyl cellulose), alginate, gelatin or cyclodextrins. Suitable polymers may also include chitosan, hyaluronic acid, xanthan, a galactomannan (such as guar gum, locust bean gum, and tara gum), gum Arabic or acacia gum, gum karaya, gum tragacanth, konjac maanan, pectin, carrageenan, gellan, or agar.

In addition to affecting viscosity of the composition, polymers can contribute to the osmotic pressure of the composition, assisting wound debridement. In addition, polymers such as polyethylene glycol, may have antimicrobial properties.

Viscosity can be fine-tuned for various applications by blending polymers of varying molecular weight, such as polyethylene glycol polymers of varying molecular weight. So, in some embodiments, the polymer comprises a first polymer and a second polymer, wherein the first polymer has a lower molecular weight than the second polymer. The first polymer may have a molecular weight less than 1000, and the second polymer may have a molecular weight of greater than 1000. In one example, the composition polymer may comprise a first polyethylene glycol polymer with a molecular weight less than 1000 (such as PEG 600) and a second polyethylene glycol polymer with a molecular weight greater than 100 (such as PEG 1500).

Preferably, the composition is a liquid (preferably a viscous liquid) or a gel. The composition may have the ability to conform to the shape of its container or flow under pressure. The composition may have the ability to support its own weight or maintain its shape. The composition may be classified as a semi-solid or semi-liquid, sharing some properties of both solids and liquids. Preferably, the composition is not a powder.

Compositions of the invention may have a viscosity (dynamic viscosity), of at least 10000 mPas at 20° C. and 1 atm, at least 50000 mPas at 20° C. and 1 atm, or at least 75000 mPas at 20° C. and 1 atm.

Compositions of the invention preferably contain less than 2% by weight of non-aqueous solvent. Even more preferably, compositions of the invention contain substantially no non-aqueous solvent. The applicant has surprisingly found that the presence of non-aqueous solvent can have a detrimental effect on the ability of compositions to generate hydrogen peroxide.

Non-aqueous solvent may include organic solvents, such as polar organic solvent. Non-aqueous solvent may include non-polymeric non-aqueous solvents such as ethanol, dimethyl sulphoxide, glycerol (or glycerin or glycerine, as it is otherwise known), ethylene glycol or propylene glycol. So, compositions of the invention may not comprise non-aqueous solvents in the form of alcohols such as polyols.

According to the invention, there is provided a composition (preferably a liquid or gel composition) comprising: enzyme that is able to convert a substrate to release hydrogen peroxide; substrate for the enzyme; and polymer, wherein the composition: a) does not comprise sufficient free water to allow the enzyme to convert the substrate; and/or b) has a water activity (a_{w}) of 0.7 or less; and/or c) comprises 5% or less, by weight, of water, and wherein the composition comprises less than 2% non-aqueous solvent, preferably substantially no non-aqueous solvent. Preferably, there is greater than 50%, by weight, of the polymer in the composition and/or less than 20%, by weight, of sugar in the composition. Most preferably, there is greater than 50%, by weight, of the polymer in the composition and less than 20%, by weight, of sugar in the composition.

According to the invention, there is provided a method of preparing a composition (preferably a liquid or gel composition) comprising mixing: enzyme that is able to convert a substrate to release hydrogen peroxide; substrate for the enzyme; and polymer, wherein the composition is formulated: a) such that there is not sufficient free water to allow the enzyme to convert the substrate; and/or b) to have a water activity of 0.7 or less; and/or c) to comprise 5% or less, by weight, of water, and wherein the method comprises adding less than 2%, by weight, of non-aqueous solvent, preferably adding substantially no non-aqueous solvent. Preferably, the polymer is added such that it is greater than 50%, by weight, of the composition and/or ii) sugar is added such that there is less than 20%, by weight, of sugar in the composition.

References herein to “enzyme” encompass one or more enzymes. For example, in some embodiments, the enzyme may comprise a plurality of enzymes that are able to convert a substrate to release hydrogen peroxide. In preferred embodiments, compositions of the invention may comprise only one enzyme that is able to convert a substrate to release hydrogen peroxide. Preferably, the only enzyme in the composition that is able to convert a substrate to release hydrogen peroxide is glucose oxidase.

Preferably, the enzyme is, or comprises, a purified enzyme. The term “purified enzyme” is used herein to include an enzyme preparation in which the enzyme has been separated from at least some of the impurities originally present when the enzyme was produced. Preferably impurities that have been removed or reduced include those that would otherwise interfere with the ability of the enzyme to convert the substrate to release hydrogen peroxide.

Preferably, the enzyme is at least 95% pure. Even more preferably, the enzyme is at least 98% pure. Most preferably, the enzyme is at least 99% pure. Preferably, the enzyme is pharmaceutical grade.

The enzyme may have been produced by recombinant or non-recombinant means, and may be a recombinant or non-recombinant enzyme. The enzyme may be purified from a microbial source, preferably from a non-genetically modified microbe.

The enzyme may be 0.0005% to 0.5% (by weight), 0.001% to 0.2% (by weight), 0.001% to 0.1% (by weight) enzyme, or 0.01% to 0.05% (by weight) of the composition. The enzyme may be 0.001% to 0.01% (by weight) of the composition.

A suitable amount of enzyme can readily be determined by a person of ordinary skill in the art, if necessary using a well diffusion assay, to determine the extent of hydrogen peroxide release for different amounts of enzyme. The amount of enzyme used may be selected so as to produce a composition for generating antimicrobial activity that is equivalent to a selected phenol standard (for example a 10%, 20%, or 30% phenol standard).

Compositions of the invention may comprise at least 1 unit, and preferably up to 1500 units, of the enzyme per gram of the composition. A “unit” is defined herein as the amount of enzyme (e.g. glucose oxidase) causing the oxidation of 1 micromole of substrate (e.g. glucose) per minute at 25 degrees centigrade at pH 7.0.

In some embodiments, the comprises more than 15 units, for example at least 30 units, at least 50 units, or at least 100 units, and suitably less than 685 units, for example 100-500 units, of enzyme (e.g. glucose oxidase) per gram of the composition. In other embodiments of the invention, a composition according to the invention comprises at least 500 units, for example 500-1000 units, or 685-1000 units, of enzyme (e.g. glucose oxidase) per gram of the composition.

Preferably, the enzyme is, or comprises, an oxidoreductase enzyme. Examples of oxidoreductase enzymes include glucose oxidase, hexose oxidase, cholesterol oxidase, galactose oxidase, pyranose oxidase, choline oxidase, pyruvate oxidase, glycollate oxidase, amino acid oxidase, or mannose oxidase.

Preferably, the oxidoreductase enzyme is, or comprises, glucose oxidase and the substrate for the oxidoreductase enzyme is, or comprises, glucose.

References herein to “substrate” encompass one or more substrates. For example, in some embodiments, the substrate may comprise a plurality of substrates. In preferred embodiments, the substrate comprises only one substrate. Preferably, the only substrate in the composition is glucose.

Preferably, the substrate is, or comprises, a purified substrate. The term “purified substrate” is used herein to include a substrate which has been separated from at least some of the impurities originally present when the substrate was obtained or produced. The purified substrate may be obtained from a natural source or may be synthetically produced. The purified substrate may be a processed, extracted, or refined substrate (i.e. a substrate in which impurities or unwanted elements have been removed by processing). Preferably, the purified substrate is at least 90%, 95%, or 99% pure (mass purity). Preferably, the substrate is pharmaceutical grade.

Preferably, the substrate is, or comprises, sugar. The term “sugar” is used herein to refer to a carbohydrate with the general formula Cₘ(H₂O)ₙ. The sugar may be obtained from a natural source (for example a processed, extracted, or refined natural sugar), or be synthetically produced. The sugar is preferably at least 90%, 95%, or 99% pure (mass purity). The sugar is preferably a pharmaceutical grade sugar. The sugar may be a monosaccharide or a disaccharide, preferably a monosaccharide. The sugar may include, for example purified D-glucose, hexose, or D-galactose. For example, the purified sugar may be medical grade, medical device grade, or pharmaceutical grade D-glucose, hexose, or D-galactose. The sugar may be an anhydrous sugar. For example, the glucose may be anhydrous glucose.

Preferably, there is 15% or less, by weight, of the substrate in compositions of the invention. There may be 10% or less, by weight, of the substrate. There may be less than 10% by weight of the substrate. Preferably there is 5% or less, by weight, of the substrate, e.g. about 3%, by weight, of the substrate.

There may be at least 1% by weight of the substrate. Preferably, there is 1% to 5%, by weight, of the substrate in the composition.

Instead of, or in addition to, the substrate, the composition may comprise a precursor substrate. Any disclosure herein which relates to the substrate, such as amounts and purity, may also apply to the precursor substrate.

For compositions of the invention which comprise a precursor-substrate, the composition may comprise one or more enzymes for converting the precursor-substrate to the substrate for the enzyme. However, in some embodiments, the precursor-substrate may not necessarily be converted to the substrate enzymatically. For example, for some precursor substrates, addition of water may be sufficient for conversion. Alternatively or additionally, compositions of the invention may comprise non-enzymatic catalysts.

Compositions which comprise a precursor-substrate may comprise a first enzyme that is able to convert the substrate to release hydrogen peroxide, and a second enzyme that is able to convert the precursor-substrate to the substrate for the first enzyme.

The precursor-substrate is preferably a carbohydrate, such as a polysaccharide, or a sugar e.g. a disaccharide, or sugar derivative. For example, the precursor-substrate may be sucrose, the first enzyme may be glucose oxidase and the second enzyme may be invertase. In another example, the precursor-substrate may be maltose, the first enzyme may be glucose oxidase and the second enzyme may be maltase.

Compositions of the invention which comprise a precursor-substrate may comprise an enzyme (preferably a purified enzyme) that is able to convert the substrate to release hydrogen peroxide, and at least two enzymes (e.g. second and third enzymes, preferably purified enzymes) that are able to convert the precursor-substrate to the substrate for the first enzyme. For example, the precursor-substrate may be starch, the first enzyme may be glucose oxidase and the second and third enzymes may be amylase and maltase. For example, the precursor-substrate may be cellulose, the first enzyme may be glucose oxidase and the second and third enzymes may be cellulose and beta-glucosidase.

Compositions of the invention preferably comprise an additional component in the form of solute. Preferably, the solute is soluble in water. References herein to “solute” encompass one or more solutes. For example, in some embodiments, the solute comprise a plurality of solutes. In preferred embodiments, the solute comprises only one solute.

The solute is preferably distinct from the substrate. For instance, in one particularly preferred embodiment, the only substrate is glucose and the only solute is fructose.

The solute is preferably purified, meaning that the solute has been separated from at least some of the impurities originally present when the solute was obtained or produced. The solute may be obtained from a natural source or may be synthetically produced. The solute may be a processed, extracted, or refined substrate (i.e. a solute in which impurities or unwanted elements have been removed by processing). Preferably, the solute is 90%, 95%, or 99% pure (mass purity). Preferably, the solute is pharmaceutical grade.

The solute may be, or may comprise, a carbohydrate. The solute may be, or may comprise, a polysaccharide. Preferably, the solute is, or comprises, sugar or sugar derivative. More preferably, the solute is, or comprises, a sugar. Suitable sugars include oligosaccharides, disaccharides or monosaccharides. Preferably, the sugar is, or comprises, a disaccharide or a monosaccharide. In particularly preferred embodiments, the sugar is, or comprises, a monosaccharide. Suitable sugars may include fructose, glucose, galactose, sucrose, maltose. In a particularly preferred embodiment, the sugar is fructose.

The solute may be, or may comprise, a sugar derivative. “Sugar derivative” is used herein to refer to a sugar that has been modified by addition of one or more substituents. The substituent may be something other than a hydroxyl group. Sugar derivatives encompasses amino sugars, acidic sugars, deoxy sugars, sugar alcohols, glycosylamines and sugar phosphates. For example, sugar derivatives may include glucose-6-phosphateglucosamine, glucoronate, gluconate, galactosamine, glucosamine, sialic acid, deoxyribosefucose, rhamnose glucuronic acid, polyols (e.g. sorbitol, erythritol, xylitol, mannitol, lactitol and maltitol) and sucralose.

The solute may comprise two or more solutes, as described herein. For example, compositions of the invention may comprise two or more sugars or sugar derivatives. The composition may comprise a maximum of two solutes, e.g. two sugars or sugar derivatives; or a maximum of three solutes, e.g. three sugars or sugar derivatives. For instance, a composition of the invention may comprise glucose, fructose and sucrose. However, in a particularly preferred embodiment of the invention, the composition comprises glucose and fructose and no other mono or disaccharides.

The solute preferably has a high solubility in water, for example a solubility which is greater than glucose. Glucose has a solubility of 90 g/100 g water at 20° C. and 1 atm. In a preferred embodiment, the solute has a solubility greater than or equal to 100 g/100 g water at 20° C. and 1 atm, in a more preferred embodiment, the solute has a solubility greater than or equal to 200 g/100 g water at 20° C. and 1 atm, in an even more preferred embodiment, the solute has a solubility greater than 300 g/100 g water at 20° C. and 1 atm.

A solute with a high solubility may assist in reducing the free water available in the final composition, or during production of the composition. A solute with a high solubility may also assist in obtaining a desired viscosity. The presence of a solute, such a fructose, may assist in reducing crystallisation, or separation, of the composition.

Fructose is a particularly preferred solute because it has a solubility of about 375 g/100 g water at 20° C. and 1 atm.

The solute (e.g. fructose) is preferably present in an amount of 10% or less, by weight, of the composition. There may be 15% or less by weight, of the solute. There may be 10% or less, by weight, of the solute. There may be less than 10% by weight of the solute. There may be 5% or less, by weight of the solute, e.g. about 3%, by weight, of the solute.

There may be at least 1% by weight of the solute. For example, compositions of the invention may have 1% to 5% by weight of the solute.

Compositions with high sugar concentrations may be unstable and may be prone to crystallisation and separation. High levels of sugar may also cause pain when administered to a wound, resulting from the high osmotic pressure generated.

Nevertheless, the applicant has appreciated that there may still be benefits in maintaining low levels of sugars. The vascular systems of patients with wounds may be compromised, meaning nutrients may not be able to reach the wound. A composition that provides sugars may thus provide the wound with an efficient energy source, avoiding the breakdown of proteins by bacteria reducing generation of odour-causing compounds such as hydrogen sulphide. Furthermore, the presence of sugars can also contribute to minimising the amount of free water in the composition, thus contributing to stability of the composition and minimising hydrogen peroxide production until it is necessary, upon dilution of the composition. Low levels of sugars may still affect the viscosity of the composition and the osmotic activity of the composition, supporting wound debridement.

In compositions of the invention, the total amount of sugar (e.g. monosaccharides and disaccharides) is preferably 10% or less by weight. Preferably the total amount of sugar is 7.5% or less, by weight. In one example, the total amount of sugar in compositions of the invention is 1% to 10% by weight. In one embodiment, the total amount of sugar in the composition is 1 to 7.5% by weight.

Compositions of the invention may contain less than 10% by weight of solute (e.g. fructose) and less than 10% by weight of substrate (e.g. glucose). Compositions of the invention may contain 1-10% by weight of solute (e.g. fructose) and 1-10% by weight of substrate (e.g. glucose). Preferably, the composition contains 1-5% by weight of solute (e.g. fructose) and 1-5% by weight of substrate (e.g. glucose).

Compositions of the invention may comprises at least two sugars or sugar derivatives (e.g. including glucose and fructose). The composition may comprise a maximum of two sugars or sugar derivatives (e.g. only glucose and fructose). However, compositions of the invention could comprise only one sugar, such as glucose.

In a preferred embodiment, the enzyme is at least 95% pure, the solute is at least 95% pure and the substrate is at least 95% pure (all with reference to mass purity).

In another preferred embodiment, the enzyme is at least 98% pure, the solute is at least 98% pure and the substrate is at least 95% pure (all with reference to mass purity).

In another preferred embodiment, the enzyme is at least 99% pure, the solute is at least 99% pure and the substrate is at least 99% pure (all with reference to mass purity).

Compositions of the invention, or components of compositions of the invention, may be pharmaceutical grade. The term “pharmaceutical grade” is used herein to refer to include reference to a purity standard for a reagent that has been established by a recognized national or regional pharmacopeia (e.g., the U.S. Pharmacopeia (USP), British Pharmacopeia (BP), National Formulary (NF), European Pharmacopoeia (EP), or Japanese Pharmacopeia (JP)).

Compositions of the invention may contain a trace amount (or low levels) of free water that may allow a trace amount (or low levels) of hydrogen peroxide to be produced.

Before dilution, hydrogen peroxide may be present at a concentration of less than 10 ppm, 6 ppm or less, 5 ppm or less, 3 ppm or less, or 2 ppm or less. Hydrogen peroxide may be present in the composition at a concentration of 120 UM or less, preferably 100 UM or less, more preferably 80 UM or less. Low levels of hydrogen peroxide before dilution may advantageously improve the shelf life of compositions of the invention. Higher levels of hydrogen peroxide may result in loss of enzyme activity over time. This may be caused by oxidative damage to the enzyme by the hydrogen peroxide being produced.

Once the composition is diluted, hydrogen peroxide may be generated at substantial concentrations. At 1 hour, following a 1:1 dilution (by weight) with water, the level of hydrogen peroxide production may increase by a factor of at least 5, at least 10, at least 20, at least 50, at least 100, at least 200 or at least 300. At 24 hours, following a 1:1 dilution (by weight) with water, the level of hydrogen peroxide production may increase by a factor of at least 5, at least 10, at least 20, at least 50, at least 100, at least 200, or at least 300.

In compositions of the invention, the water activity (a_{w}) may be 0.8 or less, 0.7 or less, 0.6 or less, 0.5 or less, 0.4 or less, or 0.3 or less. For example, the water activity may be 0.2 to 0.8, for example 0.3 to 0.7 or 0.4 to 0.6. A low water activity may be advantageous in preventing microbial proliferation, and it may be advantageous in minimising hydrogen peroxide production prior to activation by dilution, thus helping to maintain stability of the composition over an extended period of time.

Water activity is typically measured using a hygrometer, such as a resistive electrolytic hygrometer, a capacitance hygrometer or a dew point hygrometer. Measurement of water activity would typically take place at ambient temperature, such as normal temperature and pressure. Measurement of water activity may take place according to ISO 18787:2017.

Preferably, compositions of the invention contain water, but at very low levels. The presence of water may provide a solvent for the dissolution of the substrate and the optional solute.

Suitable amounts of water will vary depending on the precise components of the composition. Typically, there would be 5% (by weight) or less, preferably 1% or less (by weight) water in the composition. Preferably, there is at least 0.01%, most preferably at least 0.05% by weight of water in the composition.

Preferably, compositions of the invention do not include sufficient free water to allow the enzyme to convert the substrate. The amount of water which is “sufficient” to convert the substrate would vary and would depend on the exact composition of the formulation. The amount of free water is affected by the type and concentration of the sugars present. The threshold at which sufficient free water is present could be established by the skilled person. For example, the skilled person can vary the amount of water in a composition and determine at which point hydrogen peroxide is detected using, for instance, a Merckoquant peroxide test kit as described WO2015/166197.

Compositions of the invention preferably contain substantially no catalase.

Compositions of the invention preferably contain substantially no peroxidase.

Compositions of the invention preferably contain substantially no lactoferrin.

Compositions of the invention preferably contain substantially no zinc oxide.

Compositions of the invention preferably do not comprise honey.

Compositions of the invention preferably do not comprise antioxidant. Examples of antioxidant include ascorbic acid, tocopherol or ascorbyl palmitate. So, compositions of the invention may not comprise ascorbic acid, tocopherol or ascorbyl palmitate.

In some compositions of the invention, there is substantially no oil or other lipophilic phase. Compositions of the invention are preferably not emulsions.

The composition may comprise 75-97% (by weight) polymer, 1-5% (by weight) substrate, 0.001-0.2% (by weight) enzyme, and 0.01 to 1% water.

The composition may comprise 85-95% (by weight) polymer, 2-5% (by weight) substrate, 0.001-0.2% (by weight) enzyme, and 0.05-1% (by weight) water.

The composition may comprise 75-97% (by weight) polymer (preferably PEG), 1-5% (by weight) glucose, 0.001-0.2% (by weight) glucose oxidase, and 0.01 to 1% water.

The composition may comprise 85-95% (by weight) polymer (preferably PEG), 2-5% (by weight) glucose, 0.001-0.2% (by weight) glucose oxidase, and 0.05-1% (by weight) water.

The composition may comprise, or consists of, 75-97% (by weight) polymer (preferably PEG), 1-5% (by weight) glucose, 1-5% (by weight) fructose, 0.001-0.2% (by weight) glucose oxidase, and 0.01 to 1% water.

The composition may comprise, or consists of, 85-97% (by weight) polymer (preferably PEG), 2-5% (by weight) glucose, 2-5% (by weight) fructose, 0.001-0.2% (by weight) glucose oxidase, and 0.05-1% (by weight) water.

The polymer is preferably polyethylene glycol, and more preferably comprises two types of polyethylene glycol of different molecular weight. The first polyethylene glycol preferably has a molecular weight less than 1000 (such as PEG 600) and the second polyethylene glycol preferably has a molecular weight greater than 1000 (such as PEG 1500). There is preferably more of the lower molecular weight polyethylene glycol than the higher molecular weight polyethylene glycol.

Compositions of the invention are preferably sterile and may be sterilised by any suitable means.

Preferably compositions of the invention have been sterilised by irradiation. Irradiation may be achieved by gamma, electron beam or X-ray. The Applicant has found that compositions can retain glucose oxidase activity (and, therefore, the ability to release hydrogen peroxide on dilution) following sterilisation by exposure to gamma irradiation or electron beam irradiation. A suitable level of gamma irradiation is 10-70 kGy, preferably 25-70 kGy, more preferably 35-70 kGy. Alternatively, compositions of the invention may be sterilised by electron beam irradiation. A suitable level or dose of irradiation (e.g. electron beam irradiation) may be 10-100 kGy, preferably 30-80 kGy, more preferably 50-80 kGy. The dose may be greater than 35 kGy. The dose may be less than 80 kGy, for example 75 kGy or less. The dose may be 25-50 kGy. A suitable dose may be 10-25 kGy. A dose of 20-30 kGy may be preferred, such as about 25 kGy.

Compositions of the invention may be in a container or sachet. The container may assist in maintaining the sterility of the composition. Preferably, the container is sealed or airtight. The container may have a removable and/or replaceable cap or seal. The container is preferably opaque. The composition may be contained within a syringe or a tube. For example, the composition may be contained within a high-density polyethylene/low-density polyethylene (HDPE/LDPE) tube or in polyester-aluminium-polyethylene (PET/Al/PE) sachet.

A composition of the invention may be provided with a dressing material. The dressing material may be coated with the composition. Suitable dressings materials include gauzes, bandages, tissues, films, gels, foams, hydrocolloids, alginates, hydrogels, or polysaccharide pastes, granules, beads or tulle. It may comprise carboxymethylcellulose. The composition may be present together with a wound-dressing matrix, such as a collagen or collagen-glycosaminoglycan matrix. The dressing may be a tulle dressing. Compositions in combination with a dressing are preferably sterile, and may be sterilised using irradiation, e.g. gamma irradiation or electron beam irradiation.

Compositions of the invention can be used to treat any microbial infection that can be treated by hydrogen peroxide. Examples include infection caused by gram positive bacteria, gram negative bacteria, acid-fast bacteria, viruses, yeasts, parasitic or pathogenic micro-organisms or fungi. For example, infections caused by the following micro-organisms may be treated: Escherichia coli, Staphylococcus aureus, Pseudomonas aeruginosa, Candida albicans, Propionibacterium acnes, Staphylococcus aureus, Staphylococcus epidermidis, Staphylococcus saprophytics, Beta haemolytic Streptococci Group A or B, Campylobacter coli, Campylobacter jejuni, Methicillin Resistant Staphylococcus Aureus (MRSA), Methicillin Sensitive Staphylococcus Aureus (MSSA), Botrytis cinerea, Mycobacterium tuberculosis, Cryptosporidium, Plasmodium, Streptococcus pyogenes, Streptococcus zooepidemicus and Toxoplasma.

Compositions of the invention may treat infections caused by Candida.

There is further provided according to the invention a composition of the invention for use in the prevention or treatment of a microbial infection, for example a microbial infection that comprises a biofilm, or a microbe that is capable of forming a biofilm. So, there may be provided a composition of the invention for use in the prevention or treatment of a microbial infection that comprises a biofilm or a microbe that is capable of forming a biofilm. The biofilm may comprise bacteria, fungi and/or viruses.

There is also provided according to the invention use of a composition of the invention in the manufacture of a medicament for the prevention or treatment of a microbial infection, for example a microbial infection that comprises a biofilm, or a microbe that is capable of forming a biofilm.

The invention also provides a method of preventing or treating a microbial infection, for example a microbial infection that comprises a biofilm, or a microbe that is capable of forming a biofilm, wherein the method comprises administering an effective amount of a composition of the invention to a site of the infection.

According to the invention there is also provided use of a composition of the invention to prevent or inhibit microbial growth.

There is also provided according to the invention a composition of the invention for use as a medicament.

Compositions of the invention may be used to treat animals. Compositions of the invention may be used to treat humans.

There is further provided according to the invention a composition of the invention for the prevention, treatment, or amelioration of a microbial infection.

The invention also provides use of a composition of the invention in the manufacture of a medicament for the prevention, treatment, or amelioration of a microbial infection.

There is further provided according to the invention a method of preventing, treating, or ameliorating a microbial infection, which comprises administering a composition of the invention to a subject in need of such prevention, treatment or amelioration. The subject may be a human or animal subject. Compositions of the invention may be topically administered.

There is also provided according to the invention a device for delivering a composition to a patient, the device comprising a composition of the invention. The device may be a dispensing device containing a composition of the invention. The dispensing device may be a tube or a syringe.

According to a preferred aspect of the invention, a composition of the invention may be used in a method of wound care, including the treatment of a wound, or the treatment or management of wound sepsis.

The wound may be an acute wound, chronic wound, surgical wound (for example, a Caesarean wound), chronic burn, or an acute burn. A composition of the invention may be used in the prophylactic prevention of wound sepsis. If a storage-stable composition of the invention is used, it will be appreciated that this may be diluted by liquid present at the wound site, which thereby leads to the release of hydrogen peroxide by the diluted composition.

There is also provided according to the invention a composition of the invention for treatment of a wound. There is also provided a method of treating a wound, which comprises administering a composition of the invention to a subject in need of such treatment. There is also provided a use of a composition of the invention in the manufacture of a medicament for the treatment of a wound.

Compositions of the invention may be administered to a patient, such as placed on the wound of a patient, for a period of at least 24 hours or 48 hours or, more preferably, 72 hours.

There is further provided according to the invention use of a composition of the invention in the manufacture of a medicament for treatment of a wound.

There is also provided according to the invention a method of treating inflammation, which comprises administering a composition of the invention to a site of inflammation.

There is also provided according to the invention a composition of the invention for treatment of inflammation.

There is further provided according to the invention use of a composition of the invention in the manufacture of a medicament for treatment of inflammation.

There is also provided according to the invention a method of stimulating tissue growth, which comprises administering a composition of the invention to a site in need of such stimulation.

There is also provided according to the invention a composition of the invention for stimulating tissue growth.

There is further provided according to the invention use of a composition of the invention in the manufacture of a medicament for stimulating tissue growth.

There is also provided according to the invention a method of debriding a wound, which comprises administering a composition of the invention to a wound in need of debridement.

There is also provided according to the invention a composition of the invention for debriding a wound.

There is further provided according to the invention use of a composition of the invention in the manufacture of a medicament for debriding a wound.

There is also provided according to the invention a method of deodorising a wound, which comprises administering a composition of the invention to a wound in need of deodorising.

There is also provided according to the invention a composition of the invention for deodorising a wound.

There is further provided according to the invention use of a composition of the invention in the manufacture of a medicament for deodorising a wound.

A composition of the invention may be provided with instructions for use of the composition. For example, a composition of the invention may be packaged as a kit with the instructions.

The applicant has developed improved methods for producing compositions comprising enzyme that is able to convert a substrate to release hydrogen peroxide; substrate for the enzyme; polymer and optionally a solute i) that has solubility of at least 100 g/100 g water at 20° C. and 1 atm pressure, and/or ii) in the form of a sugar that is not glucose.

Such improved methods may allow formulation of stable compositions that require only low amounts of water, thus minimising hydrogen peroxide production prior to activation by dilution, and do not require potentially destabilising non-aqueous solvent, nor weak acidic antioxidants. For example, such improved methods may allow effective formulation of compositions as described herein.

According to the invention, there is provided a method for forming a composition, the composition comprising enzyme that is able to convert a substrate to release hydrogen peroxide; substrate for the enzyme; and polymer, wherein the composition: a) does not comprise sufficient free water to allow the enzyme to convert the substrate; and/or b) has a water activity (a_{w}) of 0.7 or less; and/or c) comprises 5% or less, by weight, of water, and optionally wherein the composition comprises a solute that has solubility of at least 100 g/100 g water at 20° C. and 1 atm pressure, and/or a solute in the form of a sugar that is not glucose. The composition may be according to the invention as described herein. The method comprises formulating a supersaturated aqueous solution comprising the substrate, and optionally the solute; and mixing the supersaturated aqueous solution with the polymer to form a substrate/polymer mixture.

Preferably, the supersaturated aqueous solution is a sugar solution. For example, the substrate may be, or comprise, a sugar. The optional solute may be, or comprise, a sugar. Preferably, the enzyme is, or comprises, glucose oxidase, the substrate is, or comprises, glucose and the optional solute is, or comprises, fructose.

The applicant has discovered that by adding substrate (such as glucose) to polymer without prior dissolution in water, the resulting compositions had an undesirable gritty texture that may be unsuitable in certain circumstances, such as for administration to wounds. This may be due to the solid substrate being held in suspension rather than in solution. By forming a supersaturated solution of the substrate, and adding the supersaturated solution to the polymer, the applicant has been able to form stable compositions with a non-gritty texture, with a minimal amount of water in the composition. The compositions formed by such a method may include droplets of supersaturated solution suspended in a polymer matrix. The droplets of supersaturated solution may be immiscible with the polymer.

Surprisingly, the applicant has also found that if the substrate is a sugar such as glucose, there may still be some risk of crystallisation of the composition. This crystallisation can be reduced by the inclusion of the solute, preferably in the form of an invert sugar, most preferably fructose.

In the supersaturated aqueous solution, the substrate may be at a concentration of at least 100 g/100 g water, preferably at least 200 g/100 g water, more preferably at least 300 g/100 g water. Preferably, the concentration of the substrate is no greater than 700 g/100 g water, more preferably no greater than 600 g/100 g water, most preferably no greater than 500 g/100 g water.

In the supersaturated aqueous solution, the optional solute may be at a concentration of at least 100 g/100 g water, preferably at least 200 g/100 g water, more preferably at least 300 g/100 g water. Preferably, the concentration of the solute is no greater than 700 g/100 g water, more preferably no greater than 600 g/100 g water, most preferably no greater than 500 g/100 g water.

The total concentration of sugar in the supersaturated aqueous solution may be at least 300 g/100 g water, preferably at least 400 g/100 g water, more preferably at least 500 g/100 g water, most preferably at least 600 g/100 g water. Preferably, the total concentration of sugar is no greater than 800 g/100 g water, more preferably no greater than 750 g/100 g water.

To allow formation of the supersaturated solution, the method may comprise dissolving at an elevated temperature, and then cooling the solution. The dissolving temperature may be at least 60° C., preferably at least 80° C., but less than 100° C. Therefore, the solution may be supersaturated at a temperature less than 60° C. The solution may be supersaturated at normal temperature and pressure (NTP).

The supersaturated solution may be mixed with liquid polymer. Before mixing, the polymer may be heated to change it from a solid phase to a liquid phase. The polymer may be heated to at least 40° C., preferably at least 50° C., and optionally to a temperature no greater than 80° C. to form the liquid polymer. The polymer may be, or may comprise, polyethylene glycol.

When mixing, the supersaturated aqueous solution and the polymer are preferably about the same temperature. For example, they may be no greater than about 5° C. difference between them, preferably no greater than about 2° C. difference. The supersaturated aqueous solution and the polymer may be at a temperature of at least 40° C., and preferably less than 80° C., more preferably less than 70° C., before mixing.

The supersaturated aqueous solution is preferably added to the polymer whilst mixing. The supersaturated aqueous solution is preferably added at a rate of not greater than 15 g per minute, more preferably not greater than 10 g per minute. When production is scaled-up, higher rates may be expected.

The method steps may thus ensure the minimisation of nucleation points and reduce the risk of crystallisation.

Mixing the supersaturated aqueous solution with the polymer may thus form the substrate/polymer mixture. The substrate polymer solution may also comprise the optional solute. Enzyme may then be added to the substrate/polymer mixture to form a substrate/polymer/enzyme mixture. Preferably, enzyme is added once the substrate/polymer mixture has cooled to less than 40° C., to minimise the risk of denaturation.

The enzyme may be pre-mixed with polymer, prior to mixing with the substrate/polymer mixture.

Preferably, the substrate/polymer/enzyme mixture is cooled whilst stirring, preferably to a temperature of less than 35° C.

According to the invention, there is provided a composition obtained or obtainable by a method of the invention.

According to the invention, there may be provided a composition wherein droplets of supersaturated aqueous solution comprising substrate and optional solute are suspended in polymer. The droplets may be dispersed within the polymer. The droplets dispersed within the polymer may be a colloid.

For example, according to the invention there is provided a composition comprising: enzyme that is able to convert a substrate to release hydrogen peroxide; substrate for the enzyme; polymer; and optional solute in the form of i) a sugar that has solubility of at least 100 g/100 g water, preferably at least 300 g/100 g water, at 20° C. and 1 atm pressure; or ii) a sugar that is not glucose, wherein the composition: a) does not comprise sufficient free water to allow the enzyme to convert the substrate; and/or b) has a water activity (a_{w}) of 0.7 or less; and/or c) comprises 5% or less, by weight, of water, and wherein droplets of supersaturated aqueous solution comprising the substrate and the optional solute are suspended in the polymer.

The enzyme may also be in the supersaturated aqueous solution.

Examples of the invention are provided in the following numbered clauses.
1. A composition, preferably a liquid or gel composition, comprising:
   enzyme that is able to convert a substrate to release hydrogen peroxide;
   substrate for the enzyme; and
   polymer
   wherein the composition: a) does not comprise sufficient free water to allow the enzyme to convert the substrate; and/or b) has a water activity (a_{w}) of 0.7 or less, and/or c) has 5% or less, by weight, of water,
   wherein there is greater than 70%, by weight, of the polymer in the composition.
2. A composition, preferably a liquid or gel composition, comprising:
   enzyme that is able to convert a substrate to release hydrogen peroxide;
   substrate for the enzyme; and
   polymer
   wherein the composition: a) does not comprise sufficient free water to allow the enzyme to convert the substrate; and/or b) has a water activity (a_{w}) of 0.7 or less; and/or c) has 5% or less, by weight, of water, and
   wherein the composition comprises less than 2%, by weight, non-aqueous solvent, and preferably i) there is greater than 50%, by weight, of the polymer in the composition; and/or ii) there is less than 20%, by weight, of sugar in the composition.
3. A composition according to clause 1, wherein there is at least 80% by weight of the polymer in the composition, preferably at least 90% by weight of the polymer in the composition.
4. A composition according to any preceding clause, wherein there is 10% or less, by weight, of sugar in the composition, preferably 6% or less, by weight, of sugar in the composition.
5. A composition according to any preceding clause, wherein there is at least 1%, by weight, of sugar in the composition
6. A composition according to any preceding clause, wherein there is 5% or less, by weight, of water in the composition, preferably less than 1%.
7. A composition according to any preceding clause, wherein there is at least 0.01%, preferably at least 0.1% by weight, of water in the composition.
8. A composition according to any preceding clause, which does not include sufficient free water to allow the enzyme to convert the substrate.
9. A composition according to any preceding clause, which has a water activity of 0.7 or less, preferably 0.6 or less.
10. A composition according to any preceding clause, wherein there is 10% or less, by weight, of the substrate in the composition, preferably 5% or less, by weight, of the substrate in the composition.
11. A composition according to any preceding clause, wherein there is at least 1%, by weight, of the substrate in the composition.
12. A composition according to any preceding clause, wherein the substrate is, or comprises, glucose and the enzyme is, or comprises, glucose oxidase.
13. A composition according to any preceding clause, comprising solute in the form of i) sugar that has solubility of at least 100 g/100 g water, preferably at least 300 g/100 g water, at 20° C. and 1 atm pressure; and/or ii) sugar that is not glucose.
14. A composition according to clause 13, wherein the solute is, or comprises, fructose.
15. A composition according to clause 13 or clause 14, wherein there is 10% or less, by weight, of the solute in the composition.
16. A composition according to any of clauses 13 to 15, wherein there is 5% or less, by weight, of the solute in the composition.
17. A composition according to any of clauses 13 to 16, wherein there is at least 1%, by weight, of the solute in the composition.
18. A composition according to any preceding clause, which comprises less than 2%, by weight, non-aqueous solvent, preferably substantially no non-aqueous solvent.
19. A composition according to any preceding clause, which comprises substantially no non-aqueous solvent in the form of ethanol, dimethyl sulphoxide, ethylene glycol or propylene glycol.
20. A composition according to any preceding clause, which comprises substantially no non-aqueous solvent in the form of an alcohol, such as glycerol.
21. A composition according to any preceding clause, which comprises substantially no antioxidant.
22. A composition according to any preceding clause, which comprises substantially no ascorbic acid, tocopherol or ascorbyl palmitate.
23. A composition according to any preceding clause, which comprises substantially no hydrogen peroxide.
24. A composition according to any preceding clause, wherein there is less than 10 ppm hydrogen peroxide in the composition.
25. A composition according to clause any preceding clause, wherein there is less than 6 ppm hydrogen peroxide in the composition.
26. A composition according to any preceding clause, wherein there is less than 3 ppm hydrogen peroxide in the composition.
27. A composition according to any preceding clause, wherein the polymer is, or comprises, polyethylene glycol.
28. A composition according to any preceding clause, wherein there is 97% or less, by weight, of the polymer in the composition, preferably 95% or less by weight.
29. A composition according to any preceding clause, wherein the polymer comprises a first polymer and a second polymer, and the first polymer has a lower molecular weight than the second polymer.
30. A composition according to clause 29, comprising a first PEG polymer of molecular weight less than 1000, a second PEG polymer of molecular weight greater than 1000.
31. A composition according to any preceding clause which is sterile.
32. A composition according to any preceding clause, wherein there is 0.001% to 0.5%, by weight, of the enzyme in the composition, preferably 0.0025% to 0.2%, by weight, of the enzyme in the composition.
33. A composition according to any preceding clause comprising substantially no honey.
34. A wound dressing which comprises a dressing material for dressing a wound, and a composition according to any preceding clause, optionally wherein the wound dressing material comprises carboxymethylcellulose.
35. A package or container comprising a composition as defined in any of clauses 1 to 33, which is air-tight or hermetically sealed.
36. A dispensing device containing a composition as defined in any of clauses 1 to 33.
37. A dispensing device according to clause 36, wherein the dispensing device is a spray, a tube or a syringe.
38. A composition according to any of clauses 1 to 33, for use as a medicament.
39. A composition according to any of clauses 1 to 33, for use in prevention, treatment or amelioration of a microbial infection.
40. A composition according to any of clauses 1 to 33, for use in treatment of a wound.
41. A method of preparing a composition, optionally wherein the composition is as defined in any of clauses 1 to 33, the method comprising mixing: enzyme that is able to convert a substrate to release hydrogen peroxide; substrate for the enzyme; and polymer,
   wherein the composition is formulated: a) such that there is not sufficient free water to allow the enzyme to convert the substrate; b) to have a water activity of 0.7 or less; and/or c) to have 5% or less, by weight, of water, and
   wherein the method comprises adding the polymer such that it is greater than 70%, by weight, of the composition.
42. A method of preparing a composition, optionally wherein the composition is as defined in clause 2 or any clause dependent on clause 2, the method comprising mixing: enzyme that is able to convert a substrate to release hydrogen peroxide; substrate for the enzyme; and polymer,
   wherein the composition is formulated wherein the composition is formulated: a) such that there is not sufficient free water to allow the enzyme to convert the substrate; b) to have a water activity of 0.7 or less; and/or c) to have 5% or less, by weight, of water, and
   wherein the method comprises adding less than 2%, by weight, of non-aqueous solvent, preferably adding substantially no non-aqueous solvent, and preferably i) adding the polymer such that it is greater than 50%, by weight, of the composition; and/or ii) adding sugar such that there is less than 20%, by weight, of the sugar in the composition.
43. A method according to clause 41 or clause 42, wherein the polymer is added such that it is at least 80% by weight of the composition, preferably at least 90% by weight of the composition.
44. A method according to any of clauses 41 to 43, wherein the composition is formulated such that there is less than 5% by weight of water in the composition, preferably less than 1%.
45. A method according to any of clauses 41 to 44, wherein the composition is formulated such that there is 10% or less, by weight, of sugar in the composition, preferably such that there is 6% or less, by weight, of sugar in the composition.
46. A method according to any of clauses 41 to 45, wherein the composition is formulated such that the water activity (a_{w}) of the composition is 0.6 or less, preferably 0.5 or less.
47. A method according to any of clauses 41 to 46, wherein the composition is formulated such that the substrate is 10% or less, by weight, of the composition.
48. A method according to any of clauses 41 to 47, wherein the composition is formulated such that the substrate is 5% or less, by weight, of the composition.
49. A method according to any of clauses 41 to 48, wherein the composition is formulated such that the substrate is at least 1%, by weight, of the composition.
50. A method according to any of clauses 41 to 49, wherein the substrate is, or comprises, glucose and the enzyme is, or comprises, glucose oxidase.
51. A method according to any of clauses 41 to 50, comprising mixing solute in the form of sugar that has solubility of at least 100 g/100 g water at 20° C. and 1 atm pressure, and/or solute in the form of sugar that is not glucose.
52. A method according to clause 51, wherein the solute is, or comprises, fructose.
53. A method according to clause 51 or clause 52, wherein the solute is added such that it is less than 20%, by weight, of the composition.
54. A method according to any of clauses 41 to 53, wherein the solute is added such that it is 10% or less, by weight, of the composition.
55. A method according to any of clauses 41 to 54, wherein the solute is added such that it is 5% or less, by weight, of the composition.
56. A method according to any of clauses 41 to 55, wherein the solute is added such that it is at least 1%, by weight, of the composition.
57. A method according to any of clauses 41 to 56, wherein substantially no non-aqueous solvent is added.
58. A method according to any of clauses 41 to 57, wherein substantially no ethanol, dimethyl sulphoxide, ethylene glycol nor propylene glycol is added.
59. A method according to any of clauses 41 to 58, wherein substantially no polyol, such as glycerol, is added.
60. A method according to any of clauses 41 to 59, wherein substantially no antioxidant is added.
61. A method according to any of clauses 41 to 60, wherein substantially no ascorbic acid, tocopherol or ascorbyl palmitate, is added
62. A method according to any of clauses 41 to 61, wherein the composition is formulated such that the composition comprises substantially no hydrogen peroxide.
63. A method according to any of clauses 41 to 62, wherein the composition is formulated such that hydrogen peroxide is present at a concentration of less than 10 ppm, less than 6 ppm or less than 3 ppm.
64. A method according to any of clauses 41 to 63, wherein the polymer is, or comprises, polyethylene glycol.
65. A method according to any of clauses 41 to 64, wherein the polymer is added such that it is 95% or less by weight of the composition.
66. A method according to any of clauses 41 to 65, wherein the polymer comprises a first polymer and a second polymer, and the first polymer has a lower molecular weight than the second polymer.
67. A method according to clause 66, wherein the composition comprising a first PEG polymer of molecular weight less than 1000, and a second PEG polymer of molecular weight of greater than 1000.
68. A method according to any of clauses 41 to 67, comprising a sterilisation step, to form a sterile composition, optionally wherein the sterilisation step comprises expose to gamma or electron beam radiation.
69. A method according to any of clauses 41 to 68, wherein the enzyme is a purified enzyme.
70. A method according to clause 69, wherein the enzyme has a mass purity at least 95%, preferably at least 98%.
71. A method according to any of clauses 41 to 70, wherein the substrate is a purified substrate.
72. A method according to clause 71, wherein the substrate has a mass purity of at least 95%, preferably at least 98%.
73. A method according to clause 71 or any clause dependent on clause 71, wherein the solute is a purified solute 74. A method according to clause 73, wherein the solute has a mass purity of at least 95%, preferably at least 98%.
75. A method according to any of clauses 41 to 74, wherein the enzyme is added such that it is 0.001% to 0.5%, by weight, of the composition, or 0.0025% to 0.2%, by weight, of the composition.
76. A method according to any of clauses 41 to 75, in which substantially no honey is added.
77. A method according to any of clauses 41 to 76, which comprises adding substantially no: zinc oxide, catalase, peroxidase, and/or lactoferrin.
78. A method: i) according to any of clauses 41 to 77, or ii) for forming a composition, the composition comprising enzyme that is able to convert a substrate to release hydrogen peroxide; substrate for the enzyme; and polymer, wherein the composition a) does not include sufficient free water to allow the enzyme to convert the substrate, b) has a water activity of 0.7 or less, and/or c) has 5% or less, by weight, of water, optionally wherein the composition comprises a solute a) that has solubility of at least 100 g/100 g water at 20° C. and 1 atm pressure, and/or b) in the form of a sugar that is not glucose,
   the method comprising:
   formulating a supersaturated aqueous solution comprising the substrate, and optionally the solute; and
   mixing the supersaturated aqueous solution with the polymer to form a substrate/polymer mixture.
79. The method according to clause 78, wherein the supersaturated aqueous solution is a sugar solution, preferably wherein the substrate is, or comprises, a sugar, and the optional solute is, or comprises, a sugar.
80. The method according to clause 78 or 79, wherein the enzyme is, or comprises, glucose oxidase, the substrate is, or comprises, glucose and the optional solute is, or comprises, fructose.
81. The method according to any of clauses 78 to 80, wherein the substrate in the solution is at a concentration of at least 100 g/100 g water, preferably at least 200 g/100 g water, more preferably at least 300 g/100 g water.
82. The method of any of clauses 78 to 81, wherein the optional solute in the solution is at a concentration of at least 100 g/100 g water, preferably at least 200 g/100 g water, more preferably at least 300 g/100 g water.
83. A method according to any of clauses 78 to 82, comprising dissolving the substrate and optional solute in water at a temperature of at least 60° C., preferably at least 80° C., but less than 100° C.; and then cooling to form the supersaturated aqueous solution.
84. A method according to any of clauses 78 to 83, wherein the polymer is or comprises polyethylene glycol.
85. A method according to any of clauses 78 to 84, wherein the polymer is heated to form a liquid polymer, prior to mixing the polymer with the supersaturated aqueous solution.
86. A method according to clause 85, wherein the polymer is heated to at least 40° C., preferably at least 50° C., and optionally to a temperature no greater than 80° C. to form the liquid polymer.
87. A method according to any of clauses 78 to 86, wherein the supersaturated aqueous solution and the polymer are at a temperature of at least 40° C., and preferably less than 80° C., more preferably less than 70° C., before mixing.
88. A method according to clause 87, wherein the temperature of the supersaturated aqueous solution is within 5° C. of the temperature of the polymer prior to mixing.
89. A method according to any of clauses 78 to 88, comprising adding the enzyme to the substrate/polymer mixture to form a substrate/polymer/enzyme mixture.
90. A method according to clause 89, wherein the enzyme is added to the substrate/polymer mixture once the substrate/polymer mixture has cooled to less than 40° C.
91. A method according to clause 90, wherein the enzyme is pre-mixed with polymer, prior to mixing with the substrate/polymer mixture.
92. A method according to any of clauses 89 to 91, wherein the substrate/polymer/enzyme solution is cooled whilst stirring, preferably to a temperature of less than 35° C.
93. A method according to any of clauses 78 to 92, wherein the polymer is greater than 50%, by weight, of the composition.
94. A method according to any of clauses 78 to 93, wherein there is less than 20%, by weight, of sugar in the composition.
95. A method according to any of clauses 78 to 94, wherein there is 10% or less, by weight, of sugar in the composition.
96. A method according to any of clauses 78 to 95, wherein there is 5% or less, by weight, of water in the composition, preferably less than 1%.
97. A method according to any of clauses 78 to 96, wherein there is at least 0.01%, by weight, of water in the composition.
98. A method according to any of clauses 78 to 97, which has a water activity of 0.6 or less, preferably 0.5 or less.
99. A method according to any of clauses 78 to 98, wherein there is 10% or less, by weight, of the substrate in the composition.
100. A method according to any of clauses 78 to, 99 wherein there is 5% or less, by weight, of the substrate in the composition.
101. A method according to any of clauses 78 to 100, wherein there is at least 1%, by weight, of the substrate in the composition.
102. A method according to any preceding clause, wherein the solute has a solubility of at least 300 g/100 g water, at 20° C. and 1 atm pressure; or ii) is a sugar that is not glucose.
103. A method according to any of clauses 78 to 102, wherein there is 20% or less, by weight, of the solute in the composition.
104. A method according to any of clauses 78 to 103, wherein there is 10% or less, by weight, of the solute in the composition.
105. A method according to any of clauses 78 to 104, wherein there is 5% or less, by weight, of the solute in the composition.
106. A method according to any of clauses 78 to 105, wherein there is at least 1%, by weight, of the solute in the composition.
107. A method according to any of clauses 78 to 106, wherein there is less than 2%, by weight, non-aqueous solvent, preferably substantially no non-aqueous solvent, in the composition.
108. A method according to any of clauses 78 to 107, wherein there is substantially no antioxidant in the composition.
109. A method according to any of clauses 78 to 108, wherein the polymer comprises a first polymer and a second polymer, and the first polymer has a lower molecular weight than the second polymer.
110. A method according to clause 109, wherein the polymer comprises a first PEG polymer of molecular weight less than 1000, a second PEG polymer of molecular weight greater than 1000.
111. A method according to any of clauses 78 to 110, which comprises a sterilisation step following formation of the substrate/polymer mixture.
112. A method according to any of clauses 78 to 111, wherein there is 0.001% to 0.5%, by weight, of the enzyme in the composition, or 0.0025% to 0.2%, by weight, of the enzyme in the composition.
113. A method according to any of clauses 78 to 112, wherein there is substantially no honey in the composition.
114. A composition obtained or obtainable by a method as defined in any of clauses 41 to 113.
115. A composition, optionally as defined in any of clauses 1 to 33, comprising:
   enzyme that is able to convert a substrate to release hydrogen peroxide;
   substrate for the enzyme;
   polymer; and
   optional solute in the form of i) a sugar that has solubility of at least 100 g/100 g water, preferably at least 300 g/100 g water, at 20° C. and 1 atm pressure; or ii) a sugar that is not glucose,
   wherein the composition does not comprise sufficient free water to allow the enzyme to convert the substrate, or has a water activity (a_{w}) of 0.7 or less, and
   wherein droplets of supersaturated aqueous solution comprising the substrate and the optional solute are suspended in the polymer.
116. A composition according to clause 115, wherein the supersaturated aqueous solution is a sugar solution, preferably wherein the substrate is, or comprises, a sugar, and the solute is, or comprises, a sugar.
117. A composition according to clause 115 or 116, wherein the enzyme is, or comprises, glucose oxidase, the substrate is, or comprises, glucose and the optional solute is, or comprises, fructose.
118. A composition according to any of clauses 115 to 117, wherein the substrate in the solution is at a concentration of at least 100 g/100 g water, preferably at least 200 g/100 g water, more preferably at least 300 g/100 g water.
119. A composition according to any of clauses 115 to 118, wherein the optional solute in the solution is at a concentration of at least 100 g/100 g water, preferably at least 200 g/100 g water, more preferably at least 300 g/100 g water.
120. A composition according to any of clauses 115 to 119, wherein the polymer is or comprises polyethylene glycol.
121. A composition according to any of clauses 115 to 120, wherein the polymer is greater than 50%, by weight, of the composition.
122. A composition according to any of clauses 115 to 121, wherein there is less than 20%, by weight, of sugar in the composition.
123. A composition according to any of clauses 115 to 122, wherein there is 10% or less, by weight, of sugar in the composition.
124. A composition according to any of clauses 115 to 123, wherein there is 5% or less, by weight, of water in the composition, preferably less than 1%.
125. A composition according to any of clauses 115 to 124, wherein there is at least 0.01%, by weight, of water in the composition.
126. A composition according to any of clauses 115 to 125, which has a water activity of 0.6 or less, preferably 0.5 or less.
127. A composition according to any of clauses 115 to 126, wherein there is 10% or less, by weight, of the substrate in the composition.
128. A composition according to any of clauses 115 to 127, wherein there is 5% or less, by weight, of the substrate in the composition.
129. A composition according to any of clauses 115 to 128, wherein there is at least 1%, by weight, of the substrate in the composition.
130. A composition according to any of clauses 115 to 129, wherein the solute has a solubility of at least 300 g/100 g water, at 20° C. and 1 atm pressure.
131. A composition according to any of clauses 115 to 130, wherein there is 20% or less, by weight, of the solute in the composition.
132. A composition according to any of clauses 115 to 131, wherein there is 10% or less, by weight, of the solute in the composition.
133. A composition according to any of clauses 115 to 132, wherein there is 5% or less, by weight, of the solute in the composition.
134. A composition according to any of clauses 115 to 133, wherein there is at least 1%, by weight, of the solute in the composition.
135. A composition according to any of clauses 115 to 134, wherein there is less than 2%, by weight, non-aqueous solvent, preferably substantially no non-aqueous solvent, in the composition.
136. A composition according to any of clauses 115 to 135, wherein there is substantially no antioxidant in the composition.
137. A composition according to any of clauses 115 to 136, wherein the polymer comprises a first polymer and a second polymer, and the first polymer has a lower molecular weight than the second polymer.
138. A composition according to clause 137, wherein the polymer comprises a first PEG polymer of molecular weight less than 1000, a second PEG polymer of molecular weight greater than 1000.
139. A composition according to any of clauses 115 to 138, which is sterile.
140. A composition according to any of clauses 115 to 139, wherein there is 0.001% to 0.5%, by weight, of the enzyme in the composition, or 0.0025% to 0.2%, by weight, of the enzyme in the composition.
141. A composition according to any of clauses 115 to 140, wherein there is substantially no honey in the composition.
142. A wound dressing which comprises a dressing material for dressing a wound, and a composition according to any of clauses 115 to 141, optionally wherein the wound dressing material comprises carboxymethylcellulose.
143. A package or container comprising a composition as defined in any of clauses 115 to 140, which is air-tight or hermetically sealed.
144. A dispensing device containing a composition as defined in any of clauses 115 to 141.
145. A dispensing device according to clause 144, wherein the dispensing device is a spray, a tube or a syringe.
146. A composition according to any of clauses 115 to 141, for use as a medicament.
147. A composition according to any of clauses 115 to 141, for use in prevention, treatment or amelioration of a microbial infection, or for use in treating a wound.

Examples are now described, with reference to the accompanying drawings in which:

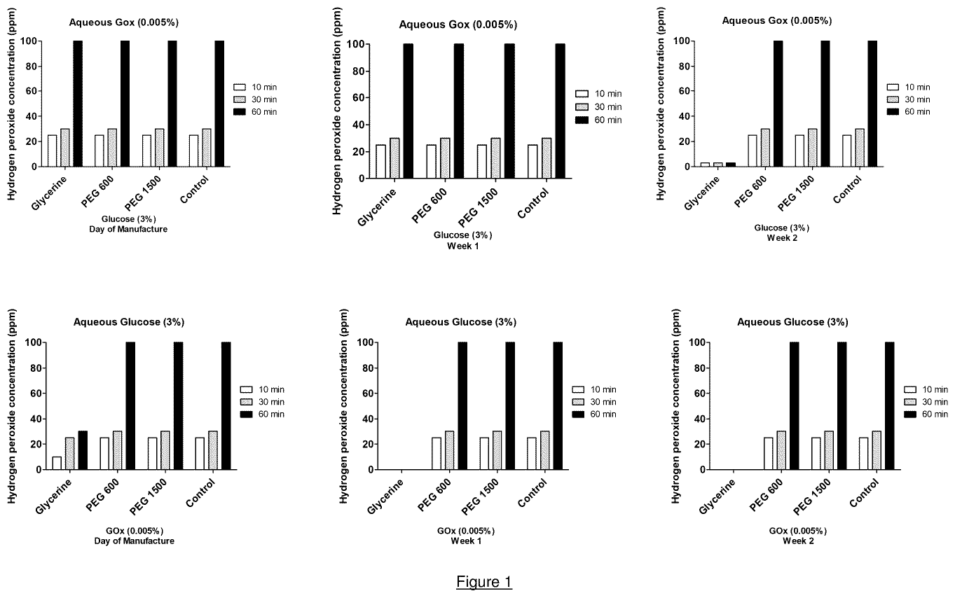

FIG. 1 shows the ability of glycerine-containing compositions and PEG-containing compositions to generate hydrogen peroxide on the day of manufacture, 1 week and 2 weeks after manufacture, compared to a positive control, following dilution;

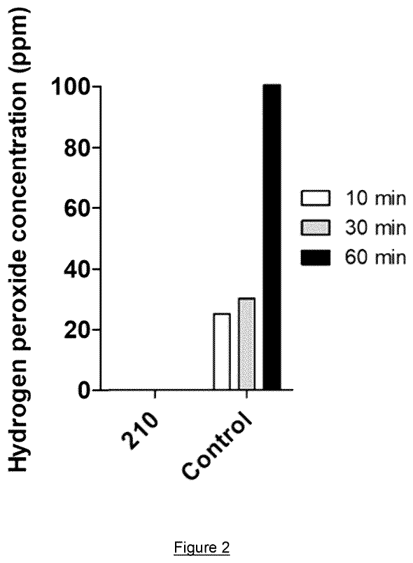

FIG. 2 shows the ability of propylene glycol-containing compositions to generate hydrogen peroxide 1 week after manufacture, following dilution;

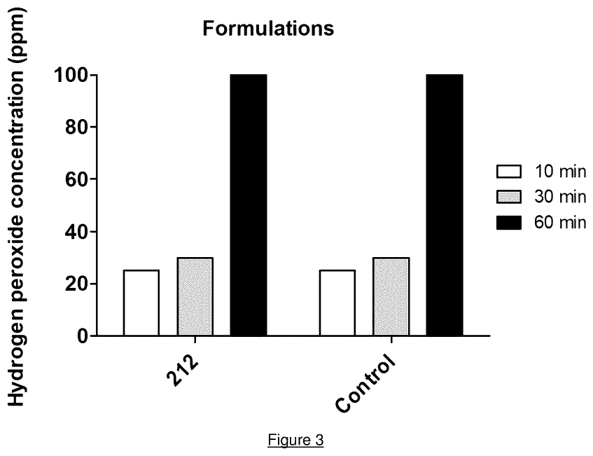

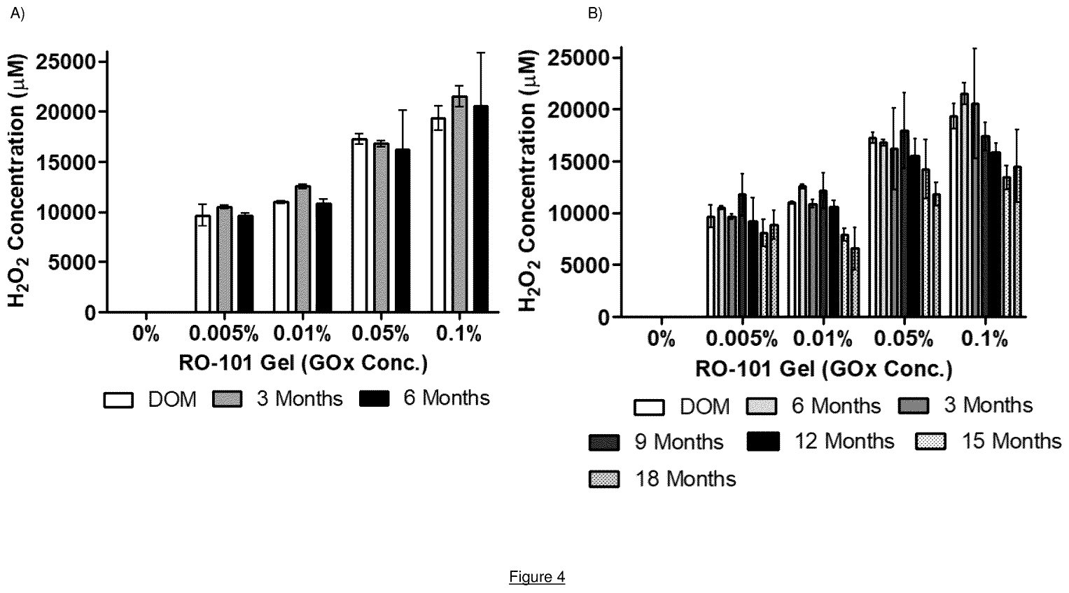

FIG. 3 shows the ability of an example of a composition of the invention to generate hydrogen peroxide, following dilution, compared to a positive control;

FIG. 4: (A) shows the ability of examples of compositions of the invention to generate hydrogen peroxide, following dilution, after storage for 3 months and 6 months; (B) shows the ability of examples of compositions of the invention to generate hydrogen peroxide, following dilution, for up to 18 months;

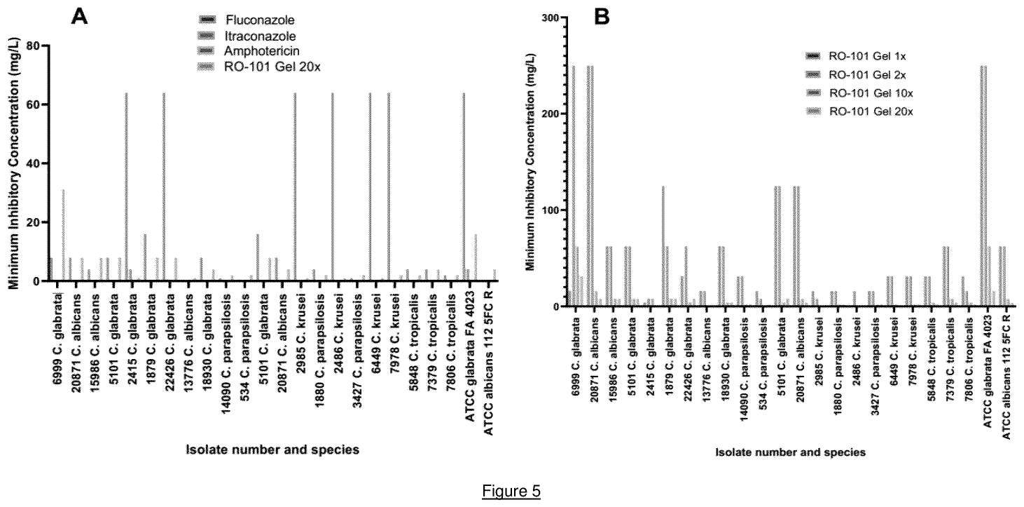

FIG. 5: (A) shows a comparison between MICs of various antifungal agents, including RO-101 gel at 20× concentration, for various Candida isolates; (B) shows a comparison between MICs of different concentrations of RO-101 Gel for various Candida isolates;

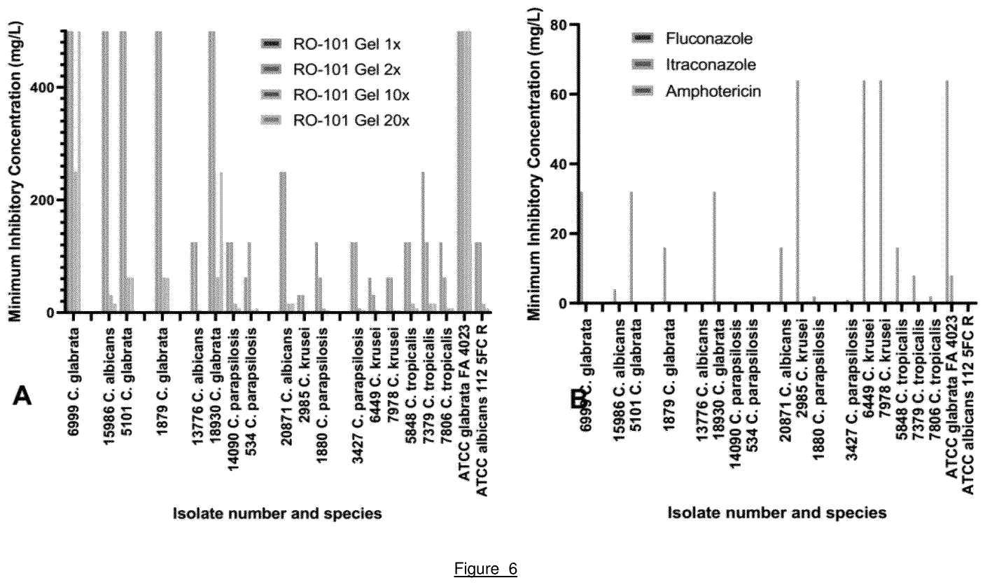

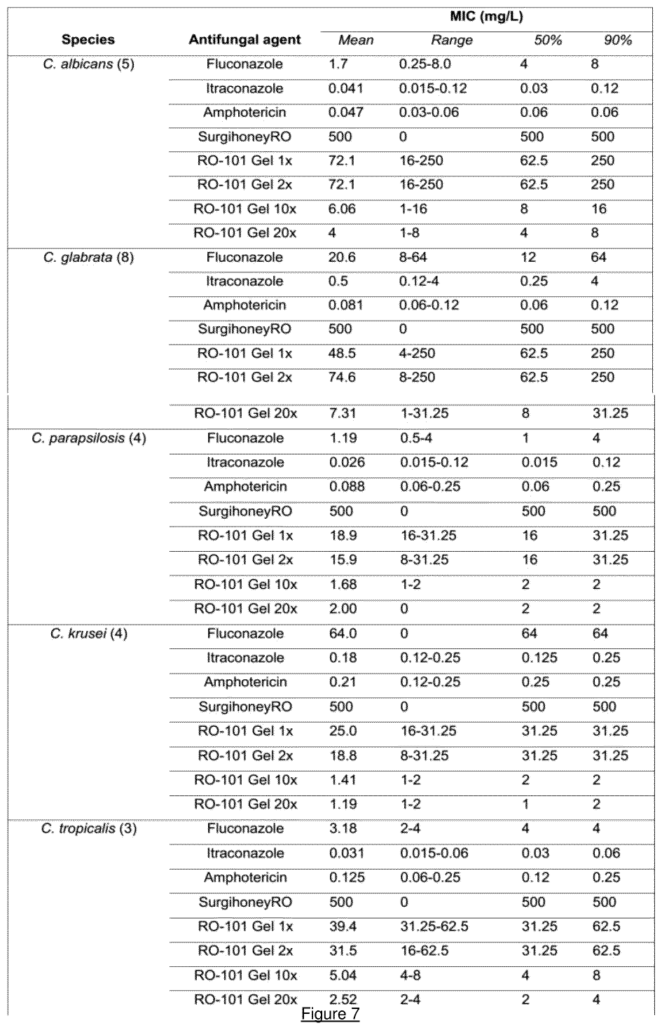

FIG. 6: (A) shows a comparison between MICs of different concentrations of RO-101 Gel for various Candida isolates after 48 hours; (B) shows a comparison between MICs of various comparator drugs after 48 hours;

FIG. 7 shows a Table of the geometric mean, range, MIC₅₀ and MIC₉₀ of various antifungal agents on various species of Candida;

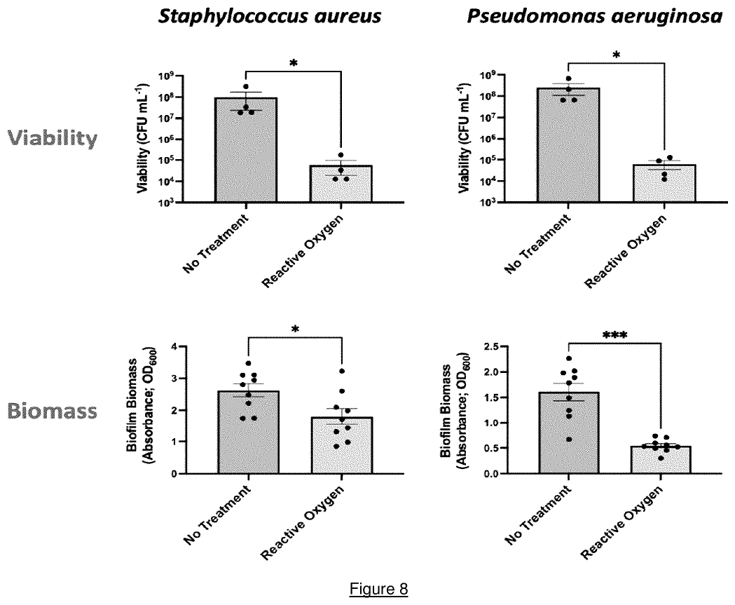

FIG. 8 shows efficacy of RO-101 gel on established biofilms. 48-hour established Staphylococcus aureus and Pseudomonas aeruginosa biofilms were treated with RO-101 gel for 24 hours. Treatment produced a significant 3-log reduction in the viability of biofilms formed by all species (measured by colony forming unit enumeration). Overall biomass of biofilms formed by S. aureus and P. aeruginosa was also significantly reduced (measured by crystal violet staining). * P≥0.05, *** P≥0.001;

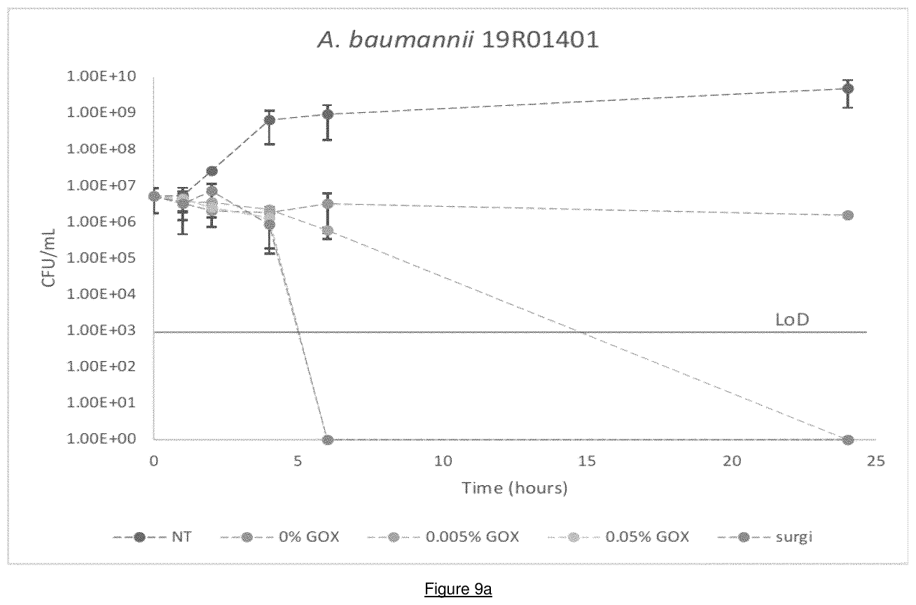

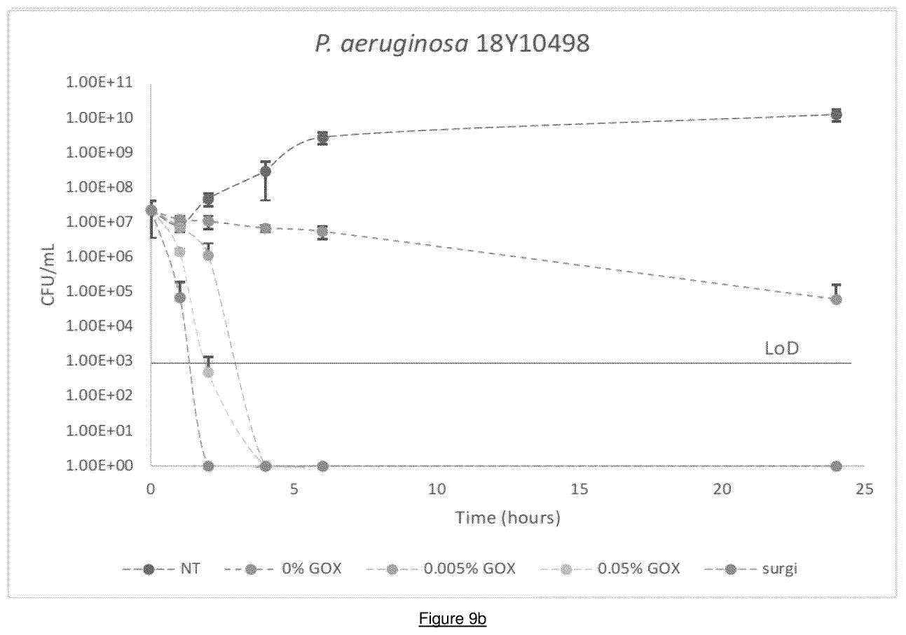

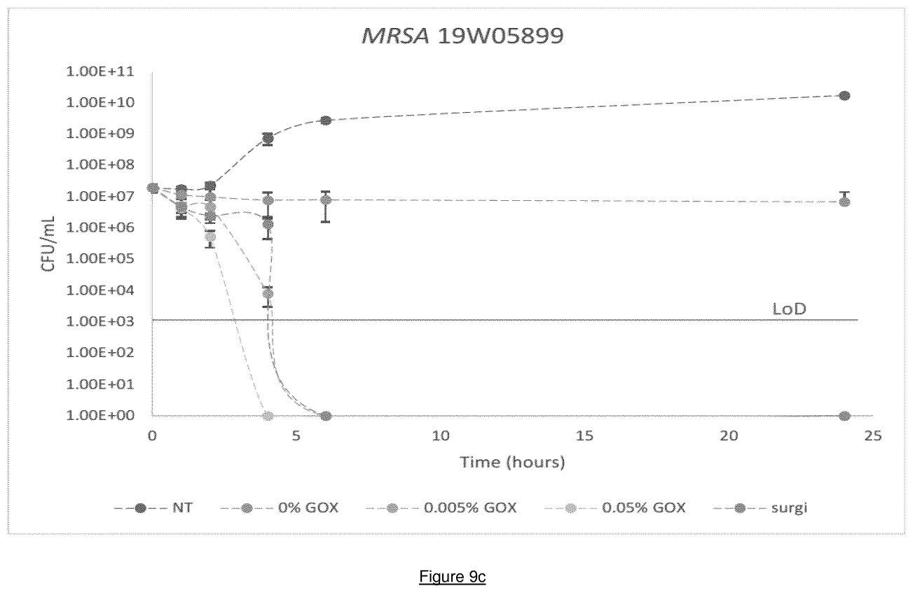

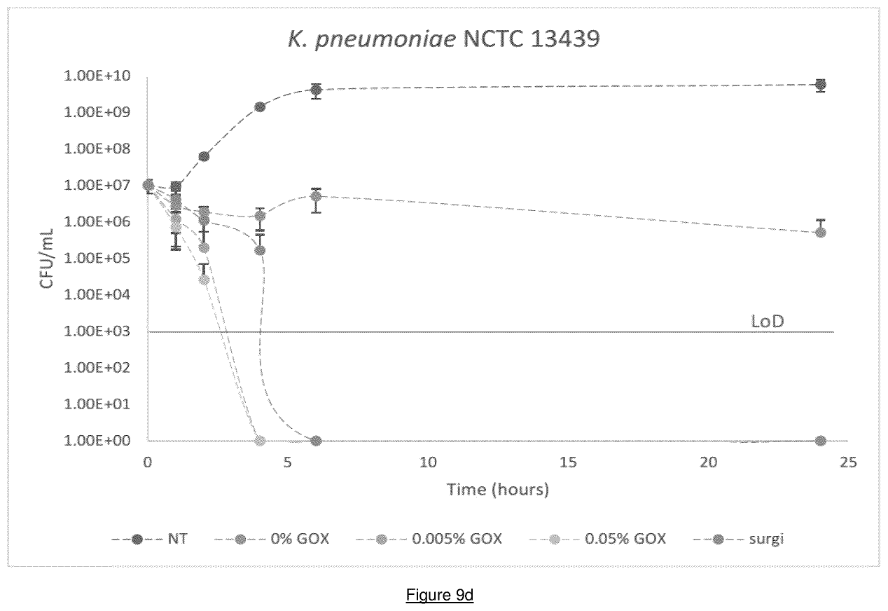

FIGS. 9 (a-d) shows time kill assays with RO-101 gel samples containing different GOX concentrations in four different clinical isolates. Bacterial cultures were inoculated with approximately 1×10⁷ CFU/mL bacteria and grown in the presence 1000 mg/ml of GOX containing RO-101 samples, the carrier control or SurgihoneyRO. An untreated control was run alongside. Viable counts were measured at each time point and the data averaged across three independent experiments. Trend lines between data points are shown as broken lines to aid interpretation. Error bars show the standard deviation of the mean.

### EXAMPLE 1

3% (w/w) glucose solutions/dispersions in either glycerine or PEG, were formulated. At the day of manufacture, after 1 week and after 2 weeks, the solutions/dispersions were mixed with glucose oxidase dissolved in water to form mixtures with a final concentration of 0.005% (w/w) glucose oxidase. Hydrogen peroxide generation was assessed using an Amplex Red assay at 10 minutes, 30 minutes and 60 minutes after activation by dilution.

0.005% glucose oxidase solutions/dispersion in either glycerine or PEG, were formulated. At the day of manufacture, after 1 week and after 2 weeks, these solutions/dispersions were mixed with glucose oxidase dissolved in water to form mixtures with a final concentration of of 3% (w/w) glucose. Hydrogen peroxide generation was assessed using an Amplex Red assay at 10 minutes, 30 minutes and 60 minutes after activation by dilution.

Hydrogen peroxide generation was assessed in comparison to a positive control of glucose and glucose oxidase dissolved in water at concentrations of 3% glucose and 0.005% glucose oxidase.

The results are shown in FIG. 1.

It was found that when aqueous solutions of glucose oxidase are added to 3% glucose/glycerine solutions, levels of hydrogen peroxide upon activation drop significantly at week 2.

It was also found that when aqueous solutions of glucose are added to 0.005% glucose oxidase/glycerine solutions, even on the date of manufacture, hydrogen peroxide levels drop significantly.

Similar results were obtained when glycerine was replaced with propylene glycol. FIG. 2 (Batch 210) shows hydrogen peroxide generation after 1 week, following addition of an aqueous solution of glucose (final concentration 3% (w/w)) to a 0.005% glucose oxidase/propylene glycol solution. Hydrogen peroxide generation was assessed using an Amplex Red assay at 10 minutes, 30 minutes and 60 minutes after activation.

Polyethylene glycol had no significant effect on the ability of the compositions to generate hydrogen peroxide.

### EXAMPLE 2

A typical specification of the excipients used for a 4 Kg batch of a composition of the invention is as follows.

| Ingredient | Quantity (g) |
|---|---|
| Glucose | 120.00 |
| Fructose | 120.00 |
| Polyethylene | 3134.76 |
| glycol (PEG) 600 | |
| Polyethylene | 600.0 |
| glycol (PEG) 1500 | |
| Glucose oxidase (GOX) | 0.20 |
| Water | 25.05 |

The list of required equipment to prepare a 4 kg batch is as follows:
2×500 mL glass beakers (1 and 3)
1×1000 mL glass beaker (2)
1×5000 mL glass vessel (4) (Vessel size will vary with batch size and should allow for sufficient headspace)
Analytical laboratory scale with a resolution of at least 0.001 g (e.g. Sartorius Cubis MSU6 microbalance)
Overhead mixer with 5 cm 4-bladed propeller stirrer (e.g. IKA RW 20 Overhead Mixer with R 1342 Propeller Stirrer.
4× hot plates
Temperature probes/thermometers
Spatulas
Weighing boats
Pipette gun
25/50 mL strippette

### Preparation of the Supersaturated Sugar Solution

Weigh an empty beaker (1) and then add 50 mL of purified water.
Set hot plate (1) to 90° C. and begin heating. As the temperature increases add alternatively and in equal proportions 180 g of glucose and 180 g of fructose.
Once the sugars are fully dissolved and a clear solution is formed turn off the hot plate (1). Weigh the contents of the beaker (to account for any evaporated water).
Calculate both the mass of the supersaturated sugar solution needed to add 3% (w/w) glucose to the final gel formulation and the total water content of the final formulation.

### Example Calculation

B=Mass of Beaker (g)
S=Mass of Solution (g)
BS=Mass of Beaker+Mass of Solution (g)
G=Mass of Glucose (g)
GR=Mass of Glucose Required (e.g. 120 g)
TGC=Percentage of glucose content
MSF=Mass of solution to be added to formulation
TWC=Total water content (g)

$$B S - B = S 495.81 g - 99.63 g = 396.18 g \qquad Eq . 1$$

$$( \frac{G}{S} ) \times 1 \; 00 = TGC ( \frac{180 g}{396.18 g} ) = 0.4 \; 543 \qquad Eq . 2$$

$$( \frac{GR}{TGC} ) = MSF \frac{120 g}{0.4543} = 264.14 g \qquad Eq . 3$$

$$MSF \times ( 1 - 2 TGC ) = TWC 264.14 g \times ( 1 - 0.9086 ) = 24.14 g \qquad Eq . 4$$

Leave beaker (1) on the hot plate (1) to reduce speed of cooling.

### Preparation of RO-101 Gel

Whilst forming the saturated sugar solution, set a second hot plate (2) to 60° C. and allow to reach temperature.
Once 60° C. is reached, add 15% (w/w) PEG 1500 to a new beaker (2) and melt for approximately 15 minutes or until a clear PEG solution is achieved.
Calculate the total percentage of the formulation thus far (glucose+fructose+water+PEG 1500+desired GOX quantity). Therefore, determine the remaining percentage (w/w) which is made up of PEG 600 (˜78%).

$$e . g . 100 \% - ( 3 \% + 3 \% + 0.6 \% + 15 \% + 0.005 \% ) = 78.395 \%$$

Set a third hot plate (3) to 60° C. and allow to reach temperature.
Weigh the desired bulk quantity of PEG 600 (as calculated at 3.3) and retain 3% in a glass beaker (3). Add the remaining quantity of PEG 600 (˜75%) to a fourth larger beaker (4) and heat on the hot plate until it reaches temperature.
Add contents of beaker (2) (PEG 1500) to contents of beaker (4) (PEG 600) and stir using the overhead mixer at 400 rpm for 2 minutes.
Adjust the hot plate temperature (3) to match the temperature of the sugar solution in beaker (1) and allow equilibrium between the hot plate and the PEG solution to be reached.
Increase the mixing speed to 600 rpm and slowly, at a rate of up to 10 g per minute, add the supersaturated sugar solution containing 3% glucose from beaker (1) (as calculated in 2.4) to the PEG mixture in beaker (4).
Maintain mixing speed for 5 minutes post addition of the supersaturated sugar solution before reducing back and maintaining a speed of 400 rpm.
Allow the mixture to cool to 35° C.
Separately mix with a spatula the retained 3% (w/w) PEG 600 in beaker (3) with the desired quantity of GOX.

### Example Calculation

BTM=Batch total mass (g)
GOR=Percentage of GOX required
GOX=Mass of GOX required

$$\frac{BTM}{100} \times GOR = GOX \frac{4000}{100} \times 0.005 = 0.2 g \qquad Eq . 5$$

Whilst stirring is maintained at 400 rpm add PEG 600/GOX solution from beaker (3) to the PEG/supersaturated sugar solution in beaker (4) to complete the formulation.
Allow the temperature to fall to 32° C. before mixing is stopped, then dispense immediately into the required storage containers.
Store at ambient temperature.

### EXAMPLE 3

A composition was formulated as follows, substantially in accordance with the method described in Example 2.

| Ingredient | % (by weight) |
|---|---|
| Glucose | 3 |
| Fructose | 3 |
| Polyethylene | 78.369 |
| glycol (PEG) 600 | |
| Polyethylene | 15 |
| glycol (PEG) 1500 | |
| Glucose oxidase | 0.005 |
| Water | 0.63 |

The resulting composition is referred to as an RO-101 gel.

The composition was assessed in comparison with the positive control described in Example 1, and was found to provide comparable hydrogen peroxide generation after 18 weeks. Hydrogen peroxide production was evaluated 10 minutes following activation, 30 minutes following activation and 60 minutes following activation by dilution with PBS. The results are shown in FIG. 3.

Hydrogen peroxide generation was assessed using an Amplex Red assay.

Similar compositions, but with varying amounts of GOX, are also referred to as RO-101 gels.

### EXAMPLE 4

Compositions formulated in accordance Example 2, but with varying levels of glucose oxidase, and sterilised using electron beam radiation at a dose of 25 kGy, were tested for their ability generate hydrogen peroxide 24 hours after activation by dilution with phosphate buffered saline (PBS) on the day of manufacture (DOM), 3 months after manufacture and 6 months after manufacture.

Hydrogen peroxide generation was assessed using an Amplex Red assay.

The results shown in FIG. 4 demonstrate that there was no significant difference between the concentration of hydrogen peroxide produced after 24 hours on the day of manufacture, after 3 months storage and after 6 months storage. The results also show that the compositions were able to maintain the ability to generate hydrogen peroxide following electron beam irradiation.

### EXAMPLE 5

The aims of this study were to establish whether RO-101 gel (of Examples 2 and 3, above) and SurgihoneyRO were able to kill strains of Candida causing VVC (Vulvovaginal Candidosis) Additionally, further aims were to compare the different enzymatic concentrations of RO-101 gel and their effectiveness in comparison with SurgihoneyRO, to ascertain whether making the product entirely synthetic would affect its efficacy.

Antifungal susceptibility testing (AST) can be performed by broth microdilution or agar-based assays, both of which are standardized by the Clinical and Laboratory Standards Institute (CLSI) and the European Committee of Antimicrobial Susceptibility Testing (EUCAST). EUCAST and CLSI set the clinical BP of different antifungal drugs (CLSI, 2022) (EUCAST, 2022a).

### Project Design

AST was used to determine a Minimum Inhibitory Concentration (MIC) value for each antifungal agent on each strain of Candida. Three comparator drugs were also tested. This allowed the effectiveness of RO-101 gel to be compared to common antifungal drugs often used to treat VVC. Antifungal agents and their concentrations were as follows: RO-101 gel (1×, 2×, 10×, 20×: 1×=0.005%, 2×=0.01%, 10×=0.05% and 20×=0.1% Gox) (1 to 500 mg/L), SurgihoneyRO (1 to 500 mg/L), RO-101 gel 0% negative control (0% Gox) (1 to 500 mg/L), Fluconazole (0.125 to 64 mg/L), Itraconazole (0.015 to 8 mg/L) and Amphotericin (0.015 to 8 mg/L). The EUCAST E.DEF 7.3.2 (EUCAST, 2022a) method for yeast susceptibility testing was followed where possible.

When using standard antifungal drugs, typically the 96-well microtiter plates with the drug dilutions and then stores the plates at −80° C. However, this was not possible for the microtiter plates loaded with the RO-101 gel, as its antimicrobial properties rely heavily on an enzyme which was activated when in contact with moisture, so once the stock solution of the gel was prepared, it was crucial that incubation with the yeast inoculums occurred quickly. This meant that the stock solutions, serial dilutions and loading of the RO-101 gel into microtiter plates had to be completed during the same period as the AST. The microtiter plates for all three comparator drugs had been preprepared and frozen.

### Media

All waste was discarded in 10% Chemgene XTM308.

Stock solutions of drugs were prepared with RPMI 1640 medium with 2% glucose. This was prepared by dissolving 20.8 g of RPMI 1640 powder (Sigma, Cat. No. R-6504), 69.1 g of morpholinopropanesulfonic acid (MOPS) (Melford, Cat. No. B2003) and 36 g of glucose (Sigma, Cat. No. G7528) in 800 ml of pharmacy water (Appleton Woods). 10M sodium hydroxide (Sigma, S8045) was added to adjust the pH to 7, and then the RPMI was filter sterilised. De Man, Rogosa & Sharpe (MRS) broth was used to grow the Lactobacillus isolates. MRS broth was prepared by dissolving 51 g of MRS powder (Sigma, 69966) in 1 litre of pharmacy water. 1 ml of Tween 80 (Sigma, P8074) was then added. The mixture was then sterilized by autoclaving at 121° C. for 15 minutes.

Sabouraud dextrose agar (SAB;Oxoid) and Columbian blood agar (CBA;Oxoid) plates were used for yeast susceptibility testing. SAB is a selective medium used for the growth of fungi, and CBA is non-selective and allows bacterial contamination to be identified. In this project, only CBA plates were used for bacterial susceptibility testing.

### Isolate Selection

Patient isolates were stored at −80° C. in a glycerol nutrient broth (GNB) solution in cryogenic vials.

All yeast isolates selected were grown from high vaginal swab samples and were of the Candida genus. Species chosen included C. albicans (n=5), C. glabrata (n=8), C. krusei (n=4), C. tropicalis (n=3) and C. parapsilosis (n=4). These isolates were identified to a species level by testing for the presence of a germ tube, biochemical testing, what temperature the isolate grew in, the colour when streaked on chrome agar, and in some cases the isolates DNA was sequenced.

The isolates were streaked for single colonies on a SAB plate, using a sterile metal loop. They were then incubated at 35° C. for 48 hours prior to susceptibility testing.

### Preparing Stock Solutions and Serial Dilutions of RO-101 Gel and SurgihoneyRO

To prepare the 1000 mg/L stock solution for each antifungal agent, 3 g of the product was diluted with 3 ml of RPMI in a bijou, and then vortexed until dissolved. 2 ml of RPMI was aliquoted into 9 other bijous. A 1:2 dilution series was then prepared by aliquoting 2 ml from each bijou into the next, until the desired concentration of 2 mg/L was acquired. This process was repeated for each antifungal agent.

100 μl of each dilution was automatic pipetted into columns 1-10 on a pre-labelled microtiter plate, with 1000 mg/L in column 1 and 2 mg/L in column 10. Columns 11 and 12 were filled with only 100 μl of RPMI. Once isolate suspensions are loaded, the concentrations of the drug dilutions will be halved due to the addition of water.

### Yeast Susceptibility Testing

The yeast susceptibility testing followed the EUCAST E.DEF 7.3.2 method (EUCAST, 2022a). Eight yeast isolates were tested each run, and one microtiter plate per drug was prepared. Two American Type Culture Collection (ATCC) strains were used as control isolates-6258 C. krusei and 22019 C. parapsilosis. These were kept constant on every microtiter plate during every run.

Before this process was started, the three comparator drug plates (Fluconazole, Itraconazole and Amphotericin) were removed from the −80° C. freezer to allow 45 minutes for defrosting before the yeast inoculums were loaded.

For each isolate, three universals were labelled 10³, 10⁵ and 10⁶, and 4.95, 13.5, and 2 ml of pharmacy water was aliquoted into each respectively. A yeast suspension was prepared in the 10⁶ universal by using a flamed wire loop to take a single colony from the preprepared SAB plate. The loop was then twisted on the side of the universal to suspend the cells, and then the universal was vortexed. The turbidity of the yeast suspension was then compared to the McFarland standards to ensure the final concentration of the yeast suspension was 1-5×10⁶ cfu/ml. The yeast suspension was then diluted 1:10 by aliquoting 1.5 ml from the 10⁶ universal into the 105 universal to be used as a working suspension. The final concentration of the working suspension should be 0.5-2.5×10⁵ cfu/ml. This was repeated for each isolate.

Using an automatic pipette, 100 μl of the working suspension was aliquoted into wells 1-11 along one row on the microtiter plate. This was repeated for each isolate on separate rows. On each plate, 100 μl of pharmacy water was aliquoted into column 12. This allowed column 11 to act as a positive control and column 12 to act as a negative control. The microtiter plates were then incubated at 35° C. and read at 24 hours and 48 hours.

The working suspension was then diluted 1:100 by aliquoting 0.05 ml into the 10³ bijou. On pre-labelled SAB and CBA plates, 10 μl of the 10³ solution was spread using a plastic hockey stick. The plates were then incubated for 48 hours. After 48 hours the colonies on the plates were counted to ensure the concentration of the working suspension had been correct. Viable counts were between 10 and 50. Also on pre-labelled SAB and CBA plates, the working suspension was streaked for single colonies using a sterile wire loop. These plates were again incubated for 48 hours. This was used to ensure there was no contamination in the working suspension. This was repeated for each isolate.

### Calculation of MIC

After 24 hours the inoculated plates were read using a Thermo Scientific Multiskan FC microdilution plate reader and the SkanIt for Multiskan FC v3.1 software. Correctly loaded plates would show maximum growth in the positive control well and no growth in the negative control well. The plates were read at 450 nm and the value of the negative control well was subtracted from the readings of the rest of the wells. The MIC for the RO-101 gels, SurgihoneyRO, Fluconazole and Itraconazole was calculated by determining the lowest drug concentration that inhibited growth by ≥50%. This was when the optical density decreased by ≥50% of the positive control well value. The same principle was applied to calculate the MIC of Amphotericin, but the lowest drug concentration to inhibit growth by ≥90%. This was calculated for each isolate on the plate. The results were then recorded.

The plate was then re-incubated and read again after 48 hours. The results were recorded.

### Statistical Analysis

For each species, descriptive statistics were performed, including a geometric mean for each antifungal agent. The MIC₅₀ and MIC₉₀ were also calculated for each antifungal agent, the MIC₅₀ being equal to the MIC value that ≥50% of the isolates are inhibited by, and the MIC₉₀ being equal to the MIC value that ≥90% of the isolates are inhibited by. The MIC₅₀ was calculated from the median of the results, and the MIC₉₀ from the 90th percentile of the results. Statistical analysis and graph production were completed on GraphPad Prism v9.4.1 software.

### Results

Of all the antifungal agents, SurgihoneyRO was the least inhibitory for all species, with the highest geometric mean (500 mg/L for all species), highest MIC₅₀ (500 mg/L for all species) and highest MIC₉₀ (500 mg/L for all species). Amphotericin was the most inhibitory for the species C. albicans and C. glabrata with the lowest geometric means (0.047 mg/L and 0.081 mg/L, respectively), lowest MIC₅₀ (0.0625 mg/L and 0.0625 mg/L, respectively) and lowest MIC₉₀ (0.0625 mg/L and 0.125 mg/L, respectively). Itraconazole was the most inhibitory for the species C. parapsilosis, C. krusei and C. tropicalis with the lowest geometric means (0.025 mg/L, 0.177 mg/L and 0.031 mg/L respectively), lowest MIC₅₀ (0.015 mg/L, 0.125 mg/L and 0.01325 mg/L, respectively) and lowest MIC₉₀ (0.125 mg/L, 0.25 mg/L and 0.0625 mg/L, respectively).

The 20× concentration of RO-101 Gel was the most inhibitory for all species, with the lowest geometric mean, MIC₅₀ and MIC₉₀. The 1× concentration of RO-101 Gel was the least inhibitory, with the highest geometric mean, MIC₅₀ and MIC₉₀. There was very little difference in the efficacy of the 20× and 10× concentration, and the same for the 1× and 2× concentration. For C. glabrata and C. parapsilosis RO-101 Gel 10× and 20× had the same MIC₅₀, however, generally the 20× concentration had a lower MIC₉₀ and smaller range.

In comparison with RO-101 Gel, both Itraconazole and Amphotericin have lower MIC₅₀, MIC₉₀ and geometric means than all of the gel concentrations. However, for C. glabrata and C. krusei species, RO-101 Gel 20× and RO-101 Gel 10× had lower MIC₅₀ and MIC₉₀ than Fluconazole, despite the C. glabrata strains being the least inhibited by RO-101 Gel. Further to this, some strains of C. glabrata and C. krusei that were resistant to Fluconazole were inhibited by RO-101 Gel (see FIG. 5).

A summary of the raw MIC data is shown in FIG. 5. MIC₅₀, MIC₉₀ and geometric mean values are displayed in FIG. 7. At 48-hours, the MIC of all the RO-101 Gel concentrations had increased, and for some isolates the growth was no longer inhibited. The MIC of the comparator drugs increased very little, if at all. The raw data for the 48-hour reads are shown in FIG. 6.

### DISCUSSION

The results from this study show that there is potential for RO-101 Gel to be used as a treatment for VVC. Each different concentration of the RO-101 Gel was able to inhibit the growth of all the species of Candida tested.

The functional properties of RO-101 Gel rely on Reactive Oxygen, mainly H₂O₂, which mimics the use of ROS in the innate immune system. The body's first line of defence against VVC is using innate immune cells to recognise yeast cells, and ROS can signal to attract monocytes and neutrophils. Further to this, ROS, such as H₂O₂, can directly damage and slow the growth of yeast cells by diffusing through their membranes. These reasons may explain the inhibitory effect RO-101 Gel has on strains of Candida causing VVC.

In comparison to strains of C. albicans, half of the strains of C. glabrata, that cause VVC, have a reduced sensitivity to Fluconazole, and there is no long-term maintenance therapy currently available. The use of RO-101 Gel as a treatment for VVC may be able to address this issue, since it has been shown the gel is able to inhibit Fluconazole resistant strains of C. glabrata. Another, greater concern in the treatment of VVC is the prevalence of Fluconazole resistant strains of C. albicans. Although this study did not use any strains of Fluconazole resistant C. albicans, the efficacy of RO-101 Gel 20× on strains of C. albicans was the same as that of Fluconazole. This suggests that there is potential for RO-101 Gel to not only replace Fluconazole as an Over-the-counter (OTC) treatment, but also be utilised for patients already infected with Fluconazole resistant strains.

This study shows that RO-101 Gel is more effective for inhibiting the growth of Candida than SurgihoneyRO.

### EXAMPLE 6

### Bacterial Strains and Media

Staphylococcus aureus strains (ATCC6538, NCTC11939, NCTC10442, and a chronic rhinosinusitis clinical isolate), Pseudomonas aeruginosa strains (NCTC6749, and cystic fibrosis clinical isolates 30, 66 and 68), and Acinetobacter baumannii strains (18X50606, 19R01401, 15P01553, and 19B00950) were all grown from 20% glycerol stocks stored at −80° C. S. aureus strains were grow using Brain Heart Infusion (BHI) agar and broth, whereas P. aeruginosa and A. baumannii strains were grown using Luria-Bertani Miller (LB) agar and broth. All strains were plated using their respective agars and incubated overnight at 37° C.

### Biofilm Viability

Bacterial broth cultures were grown overnight (˜18 h) at 37° C. under rotation (150 rpm), then diluted 1,000-fold to ˜1×10⁵ cfu/mL. One millilitre of bacterial culture was added to 12-well plates and supplemented with 1 mL ⅕th strength media diluted in dH2O. Plates were incubated under static conditions for 24 h at 37° C., 1 mL spent media replaced with 1 mL fresh ⅕th strength media, and then incubated for a further 24 h. Biofilms were washed twice with 1 mL Hanks' Balanced Salt Solution (HBSS) to remove unattached cells. One millilitre of RO-101 gel at 1,000 mg/mL was added and incubated for 24 h at 37° C., including an HBSS ‘no treatment’ control. Wells were washed twice with HBSS and remaining biofilms removed through cell scraping into 1 mL HBSS. Bacterial suspensions were then serial diluted, spot-plated onto appropriate media plates, incubated at 37° C. overnight, and colonies enumerated. Results are shown in FIG. 8.

### Biofilm Biomass

Bacterial broth cultures were grown overnight (˜18 h) at 37° C. under rotation (150 rpm), then diluted 1,000-fold to ˜1×10⁵ cfu/mL. One hundred microlitres of bacterial culture was used to inoculate flat-bottomed 96-well plates and supplemented with 100 μL ⅕th strength media diluted in dH2O. Plates were incubated under static conditions for 24 h at 37° C., 100 μL spent media replaced with 100 μL fresh ⅕th strength media, and then incubated for a further 24 h. Two hundred microlitres of RO-101 gel at 1,000 mg/mL was added and incubated for 24 h at 37° C., including an HBSS ‘no treatment’ control. Biofilms were washed twice with 200 μL HBSS then stained with 0.1% crystal violet at room temperature for 20 min. The crystal violet stain was removed, the wells washed four times with HBSS, then allowed to air-dry. Once dry, the crystal violet was solubilised by adding 200 μL 30% acetic acid for 15 min, transferred to another 96-well plate and the absorbance (OD600) measured using a Spectramax plus 384 spectrophotometer (Molecular Devices, USA). Results are shown in FIG. 8.

### Confocal Microscopy

Bacterial broth cultures were grown overnight (˜18 h) at 37° C. under rotation (150 rpm), then diluted 1,000-fold to ˜1×10⁵ cfu/mL. One millilitre of bacterial culture was added to black, clear-bottomed, poly-D-lysine coated 12-well vision plates and supplemented with 1 mL ⅕th strength media diluted in dH2O. Plates were incubated under static conditions for 24 h at 37° C., 1 mL spent media replaced with 1 mL fresh ⅕th strength media, and then incubated for a further 24 h. Biofilms were washed twice with 1 mL Hanks' Balanced Salt Solution (HBSS) to remove unattached cells. One millilitre of RO-101 gel treatments at 1,000 mg/mL were added and incubated for 24 h at 37° C., including an HBSS ‘no treatment’ control. Biofilms were stained with 500 μL Live/Dead stain (3 μL Syto 9 and 3 μL propidium iodide per 1 mL HBSS) in the dark for 15 min, washed three times with 1 mL HBSS, then covered with 1 mL 60% glycerol (prepared in dH₂O). Biofilms were imaged with a Zeiss LSM900 confocal microscope, using a ×63 oil immersion objective, and generating z-stacks with 1 μm sections.

48-hour established Pseudomonas aeruginosa and Acinetobacter baumannii biofilms were treated with RO-101 gel for 24 hours and imaged using confocal microscopy with Live/Dead staining. Three dimensional images were taken using 1 μm z-stack sections. Treatment of biofilms formed by both species, with RO-101 gel, resulted in a reduction in viability, represented in a reduction in the viable population relative to the control (shown as green in the image) and increase in the dead population relative to the control (shown as red in the image).

### EXAMPLE 7

### Time Kills

Bacteria were treated with compounds at 1000 mg/mL (1:1). Compounds were added to MH2 medium in a glass universal to a final volume of 3 mL containing the bacteria at 1×10⁷ CFU/mL. Assays were incubated in a shaking incubator at 200 rpm, 37° C. Time points were taken at 0, 1, 2, 4, 6 and 24 hours and a modified Miles-Misra assay was performed to determine the cell counts. In the modified Miles-Misra assay, 20 μL samples from the time kills were added to 180 μL of neutralisation buffer (as above), mixed and serially diluted and 10 μL was spotted onto TSA plates and incubated overnight, Colonies were counted and back corrected to express the data as CFU/mL. The compounds were defined as bactericidal where there was a greater than 3-log kill compared to the zero-hour time point, and bacteriostatic if there was less than, or equal to a 3-log kill.

### Results and Discussion

### Antimicrobial Efficacy Testing

Three samples of RO-101 gel (batch number 212) were provided for evaluation: two samples contained either 0.005% or 0.05% GOX; and a carrier control sample containing 0% GOX. SurgihoneyRO was also provided for evaluation alongside. The samples were tested against two panels of well characterised reference strains provided by UKHSA and a selection of current MDR (multidrug-resistant) clinical isolates generated from Hampshire Hospitals NHS Trust. The first panel is comprised of Gram-positive strains commonly associated with skin and soft tissue injuries (SSTIs) and chronic wounds. The second panel consists of a selection of Gram-negative priority pathogens including carbapenem resistant isolates. Although these Gram-negative organisms are less commonly associated with SSTIs and wounds, when they do arise, they are a much greater challenge to treat due to their multidrug resistance profiles.

MICs were determined using the CLSI standard broth microdilution method, modified to reflect the specific characteristics of the RO-101 gel and SurgihoneyRO. MBCs (minimum bactericidal concentrations) were also assessed from the MIC plate for a selection of 12 strains by inoculating TSA plates from wells adjacent to the MIC point and assessing growth after overnight incubation.

### Time Kill Data

Whilst the MIC and MBC values are useful indicators of the efficacy of the RO-101 formulation, they fail to address the situation where the actual biocidal product (hydrogen peroxide) is generated continuously within the system, as is the case for samples containing GOX. To provide additional information on the relative efficacy of the different formulations against particular species and to understand the kinetics of bacterial kill provided by samples containing GOX, time kill studies were carried out. Four current clinical isolates were selected for the work, focusing on Acinetobacter baumannii, Pseudomonas aeruginosa, Klebsiella pneumoniae and MRSA. All strains were tested with the highest dilution of formulation possible, i.e. a 1;1 dilution of compound to media (1000 mg/ml). This one concentration was used across all strains, independent of their specific MICs, to reflect the way the formulations would be used as topical treatments in the clinic, i.e. at one concentration.

In all cases, SurgihoneyRO gave a rapid bactericidal kill with the bacterial count reduced to below the limit of detection (LoD) (1×10³ CFU/mL) of the method within 6 hours for A. baumannii, K. pneumoniae and MRSA, and actually in under 4 hours for P. aeruginosa. The RO-101 samples containing 0.005% GOX also achieved a rapid bactericidal kill in 4 hours with P. aeruginosa and K. pnuemoniae and in 6 hours in MRSA. In A. baumannii however, the kill was slower, with below LoD being achieved somewhere between 6 hours and 24 hours. The RO-101@ samples containing 0.05% GOX achieved below LoD kill within 4 hours in all species tested (see FIGS. 9a-9d.).

The carrier control samples (0% GOX) achieved a growth inhibition, or bacteriostatic, profile in all strains tested.

There were no examples where there was a recovery in the viable count at 24 hours, suggesting that there is a limited number or no resistant bacteria at this time point.

### CONCLUSIONS

The data demonstrate the broad-spectrum activity of the RO-101 samples containing GOX against a range of pathogens associated with both wound infections and high levels of multidrug resistance. The MIC/MBC values demonstrate the antimicrobial efficacy of the formulations, provide an indication of the relative activity of different preparations and provide an easily measured reference for batch release of product. A range of resistance mechanisms, associated with resistance to various antibiotics including carbapenem resistance, did not impact on efficacy in the strains tested. Time kill data suggests that the formulations are rapidly bactericidal against all species tested and there was no evidence of bacterial survival or emergence of resistance within 24 hours of treatment.

### EXAMPLE 8

Typical specification of the excipients used for a 2.5 Kg batch is:

| Ingredient | Quantity (g) |
|---|---|
| Glucose | 50.00 |
| Fructose | 50.00 |
| Polyethylene | 2015.70 |
| Glycol (PEG) 600 | |
| Polyethylene | 375.00 |
| Glycol (PEG) 1500 | |
| Glucose Oxidase | 0.13 |
| Deionised water | 9.17 |

The list of required equipment to prepare a 2.5 kg batch is as follows:
1×500 mL glass beakers (1)
1×1000 mL glass beaker (2)
1×100 mL glass beaker (3)
1×4000 mL glass vessel (4) (Vessel size will vary with batch size and should allow for sufficient headspace)
Analytical laboratory scale with a resolution of at least 0.001 g (e.g. Sartorius Cubis MSU6 microbalance)
Overhead mixer with 5 cm 4-bladed propeller stirrer (e.g. IKA RW 20 Overhead Mixer with R 1342 Propeller Stirrer.
3× hot plates
Temperature probes/thermometers
Spatulas
Weighing boats
Pipette gun
25/50 mL strippette
Water bath

### Preparation of the Supersaturated Sugar Solution

Weigh an empty beaker (1) and then add 50 mL of deionised water.
Set hot plate (1) and begin heating until a solution temperature of 90° C. is reached. As the temperature of the solution increases add spatula wise and alternatively in equal proportions 180 g of glucose and 180 g of fructose.
Once the sugars are fully dissolved and a clear solution is formed turn off the hot plate (1). Weigh the contents of the beaker (to account for any evaporated water).
Calculate both the mass of the supersaturated sugar solution needed to add 2% (w/w) glucose to the final gel formulation and the total water content of the final formulation.

### Example Calculation:

B=Mass of Beaker (g)
S=Mass of Solution (g)
BS=Mass of Beaker+Mass of Solution (g)
G=Mass of Glucose (g)
GR=Mass of Glucose Required (e.g. 50 g)
TGC=Percentage of glucose content
MSF=Mass of solution to be added to formulation
TWC=Total water content (g)

$$B S - B = S 495.81 g - 99.63 g = 396.18 g \qquad Eq . 1$$

$$( \frac{G}{S} ) \times 1 \; 00 = TGC ( \frac{180 g}{396.18 g} ) = 0.4 \; 543 \qquad Eq . 2$$

$$( \frac{GR}{TGC} ) = MSF \frac{50 g}{0.4543} = 110.06 g \qquad Eq . 3$$

$$MSF \times ( 1 - 2 TGC ) = TWC 110.06 g \times ( 1 - 0.9086 ) = 10.06 g \qquad Eq . 4$$

Leave beaker (1) on the hot plate (1) to reduce speed of cooling.

### Preparation of RO-101 Gel.

Whilst forming the saturated sugar solution, set a second hot plate (2) to 80° C. and add 15% (w/w) PEG 1500 to a new beaker (2) and melt until a clear PEG solution is achieved.
Calculate the total percentage of the formulation thus far (glucose+fructose+water+PEG 1500+desired GOX quantity). Therefore, determine the remaining percentage (w/w) which is made up of PEG 600 (˜80%).

$$e . g . 100 \% - ( 3 \% + 3 \% + 0.4 \% + 15 \% + 0.005 \% ) = 80.6 \%$$

Weigh the desired bulk quantity of PEG 600 (as calculated at 3.3) and retain 3% in a glass beaker (3).
Set a third hot plate (3) to 80° C. and add the remaining quantity of PEG 600 (˜77%) to a fourth larger beaker (4) and heat on the hot plate until it reaches temperature.
Add the melted contents of beaker (2) (PEG 1500) to contents of beaker (4) (PEG 600).
Transfer the beaker containing the PEG mixture (4) to a water bath set at 32° C. and stir using the overhead mixer at 1500 rpm for 5 minutes.
Dropwise add the supersaturated sugar solution containing 2% glucose from beaker (1) (as calculated in 2.4) to the PEG mixture in beaker (4).
Maintain mixing speed post addition of the supersaturated sugar solution and allow the mixture to cool to 35° C.
Separately mix with a spatula the retained 3% (w/w) PEG 600 in beaker (3) with the desired quantity of GOX.

### Example Calculation:

BTM=Batch total mass (g)
GOR=Percentage of GOX required
GOX=Mass of GOX required

$$\frac{BTM}{100} \times GOR = GOX \frac{2500}{100} \times 0.005 = 0.1 \; 25 g \qquad Eq . 5$$

Whilst stirring is maintained at 1500 rpm add the PEG 600/GOX solution from beaker (3) to the PEG/supersaturated sugar solution in beaker (4) to complete the formulation.
Allow the temperature to fall to 32° C. before mixing is stopped, then dispense immediately into the required storage containers.
Store at ambient temperature.

The resulting composition is as follows:

| Ingredient | % (by weight) |
|---|---|
| Glucose | 2 |
| Fructose | 2 |
| Polyethylene | 80.628 |
| glycol (PEG) 600 | |
| Polyethylene | 15 |
| glycol (PEG) 1500 | |
| Glucose oxidase | 0.005 |
| Water | 0.37 |

Reduced amounts of glucose and fructose in this variation of the RO-101 gel, compared to the RO-101 gel of Examples 2 and 3, may lead to a reduced risk of crystallisation during manufacture.

1. A liquid or gel composition, comprising:
enzyme that is able to convert a substrate to release hydrogen peroxide;
substrate for the enzyme; and
polymer;
wherein the composition: a) does not comprise sufficient free water to allow the enzyme to convert the substrate; and/or b) has a water activity (a_{w}) of 0.7 or less; and/or c) comprises 5% or less, by weight, of water, and wherein:
A) droplets of supersaturated aqueous solution comprising the substrate are suspended in the polymer; and/or
B) there is greater than 70%, by weight, of the polymer in the composition; and/or
C) the composition comprises less than 2%, by weight, non-aqueous solvent, and i) there is greater than 50%, by weight, of the polymer in the composition; and/or ii) there is less than 20%, by weight, of sugar in the composition.

2. A composition according to claim 1, wherein there is 95% or less, by weight, of the polymer in the composition.

3. A composition according to claim 1 or claim 2, wherein the polymer is, or comprises, polyethylene glycol.

4. A composition according to any preceding claim, wherein there is 10% or less, by weight, of sugar in the composition.

5. A composition according to any preceding claim, wherein there is 10% or less, by weight, of the substrate in the composition.

6. A composition according to any preceding claim, wherein the substrate is, or comprises, glucose and the enzyme is, or comprises, glucose oxidase.

7. A composition according to any preceding claim, comprising solute in the form of i) sugar that has a solubility of at least 100 g/100 g water, preferably 300 g/100 g water, at 20° C. and 1 atm; or ii) sugar other than glucose.

8. A composition according to claim 7, wherein the solute is, or comprises, fructose.

9. A composition according to any preceding claim, which comprises substantially no non-aqueous solvent.

10. A composition according to any preceding claim, which comprises substantially no antioxidant.

11. A composition according to any preceding claim which is sterile.

12. A composition according to any of claims 1 to 11, for use as a medicament.

13. A composition according to any of claims 1 to 11, for use in prevention, treatment or amelioration of a microbial infection, or for use in treatment of a wound.

14. A method of preparing a liquid or gel composition as defined in any of claims 1 to 11, the method comprising mixing: the enzyme that is able to convert a substrate to release hydrogen peroxide; the substrate for the enzyme; and the polymer,
wherein the composition is formulated: a) such that there is not sufficient free water to allow the enzyme to convert the substrate; and/or b) to have a water activity of 0.7 or less; and/or c) to have 5% or less, by weight, of water, and wherein the method comprises:
A) formulating a supersaturated aqueous solution comprising the substrate, and mixing the supersaturated aqueous solution with the polymer to form a substrate/polymer mixture; and/or
B) adding the polymer such that it is greater than 70%, by weight, of the composition; and/or
C) adding less than 2%, by weight, of non-aqueous solvent, preferably adding substantially no non-aqueous solvent, and preferably i) adding the polymer such that it is greater than 50%, by weight, of the composition; and/or ii) adding sugar such that there is less than 20%, by weight, of the sugar in the composition.

15. A method according to claim 14, wherein the enzyme, substrate and solute have a mass purity at least 95%, preferably at least 98%.

16. A method: i) according to claim 14 or claim 15, or ii) for forming a composition comprising enzyme that is able to convert a substrate to release hydrogen peroxide; substrate for the enzyme; and polymer, wherein the composition: a) does not comprise sufficient free water to allow the enzyme to convert the substrate; and/or b) has a water activity (a_{w}) of 0.7 or less; and/or c) comprises 5% or less, by weight, of water, and optionally wherein the composition comprises solute: a) that has solubility of at least 100 g/100 g water at 20° C. and 1 atm pressure; and/or or b) in the form of a sugar that is not glucose, the method comprising:
formulating a supersaturated aqueous solution comprising the substrate, and optionally the solute; and
mixing the supersaturated aqueous solution with the polymer to form a substrate/polymer mixture.

17. The method according to claim 16, wherein the supersaturated aqueous solution is a sugar solution, preferably wherein the substrate is, or comprises, a sugar, and the optional solute is, or comprises, a sugar.

18. The method according to claim 16 or claim 17, wherein the enzyme is, or comprises, glucose oxidase, the substrate is, or comprises, glucose and the optional solute is, or comprises, fructose.

19. The method according to any of claims 16 to 18, wherein the substrate in the solution is at a concentration of at least 100 g/100 g water, preferably at least 200 g/100 g water, more preferably at least 300 g/100 g water.

20. A method according to any of claims 16 to 19, comprising dissolving the substrate and optional solute in water at a temperature of at least 60° C., preferably at least 80° C., but less than 100° C.; and then cooling to form the supersaturated aqueous solution.

21. A method according to any of claims 16 to 20, wherein the supersaturated aqueous solution and the polymer are at a temperature of at least 40° C., and preferably less than 80° C., more preferably less than 70° C., before mixing.

22. A method according to claim 21, wherein the temperature of the supersaturated aqueous solution is within 5° C. of the temperature of the polymer, prior to mixing.

23. A method according to claim 22, wherein the enzyme is added to the substrate/polymer mixture once the substrate/polymer solution has cooled to less than 40° C.

24. A composition obtained or obtainable by a method as defined in any of claims 16 to 23.
